(19) United States
(12) Patent Application Publication
(10) Pub. No.: US 2021/0113155 A1
(43) Pub. Date: Apr. 22, 2021

(54) EXAMINATION APPARATUS FOR MEDICAL EXAMINATION OF AN ANIMAL

(71) Applicant: Boehringer Ingelheim Vetmedica GmbH, Ingetheim am Rhein (DE)

(72) Inventors: Urs ANLIKER, Frauenkappelen (CH); Marco BURGENER, Bern (CH); Christian KAUTH, Villars-sur-Glâne (CH); Pascal Manuel LOSER, Liebefeld (CH); Michel Joseph SAINT-GHISLAIN, Duedingen (CH); Beat WYSS, Niederbipp (CH); Jeannine FLETH-JAMES, Mainz (DE); Reinhard FORBERGER, Ingelheim am Rhein (DE); Silke HAAG-DIERGARTEN, Frankfurt am Main (DE); Dagmar POLOTZEK, Frankfurt am Main (DE); Daniela Katharina RAHMEL, Ockenheim (DE); Tanja Margrit ZIMMERING, Heidesheim am Rhein (DE)

(73) Assignee: Boehringer Ingelheim Vetmedica GmbH, Ingelheim am Rhein (DE)

(21) Appl. No.: 17/070,968

(22) Filed: Oct. 15, 2020

(30) Foreign Application Priority Data
Oct. 17, 2019 (EP) 19 203 832.1
Oct. 17, 2019 (EP) 19203875.0

(51) Int. Cl. A61B 5/00; A61B 5/0205; A61B 5/026; A61B 5/0295; A61B 5/0408

(52) U.S. Cl. CPC A61B 5/6892; A61B 5/0205; A61B 5/0261; A61B 5/0295; A61B 5/021; A61B 2503/40; A61B 2562/0238; A61B 2562/066; A61B 5/04085

(57) ABSTRACT

An examination apparatus for medical examination, in particular determination of a blood pressure, of an animal, in particular an animal having a paw, particularly preferably an animal from the subfamily of the Felinae. The examination apparatus has a sensor device for the optical examination of an arterial blood flow of the animal, in particular for performing a photoplethysmography. For this purpose, the sensor device has at least one emitter for the emission of electromagnetic radiation and at least one detector for the detection of the radiation emitted by the emitter. The sensor device preferably has several emitters and several detectors that are arranged in a periodic structure. Alternatively or additionally, the sensor device has a limiting device which defines a border of a detection region of the sensor device so that a distance of the border from the sensor device is more than 0.5 mm and/or less than 5 mm.

### BACKGROUND OF THE INVENTION

### Field of the Invention

The present invention relates to an examination apparatus for medical examination of an animal, and a method for medical examination of an animal as well as a method of using the examination apparatus.

Generally, it is an aim of the present invention to enable or simplify a noninvasive blood pressure measurement in pets, such as cats or dogs. In humans, an inflatable cuff, which is placed around the arm, is often used for non-invasive blood pressure measurement. However, measuring blood pressure with a cuff is problematic for dogs, and in particular for cats, because these animals are not used to such examinations, and in particular for cats, it can thus be difficult to put on a cuff. On the other hand, the application of a cuff is also associated with stress for the animal, which should be avoided if possible, as the stress can falsify the result of the measurement.

However, the present invention is not limited to the application to pets such as cats or dogs, but can in principle be used for any kind of animal, in particular humans as well. Furthermore, the present invention is not limited to a blood pressure measurement, but is generally designed or suitable for medical examination, in particular an optical, non-invasive and/or percutaneous examination, particularly preferably photoplethysmography (PPG) and/or pulse oximetry.

### Description of Related Art

In addition to a blood pressure measurement using a cuff, other methods for non-invasive determination of blood pressure are already known in the prior art.

International Patent Application Publication WO 85/03211 A1 relates to a method for determining the arterial blood pressure, in which heartbeats are measured by means of an electrocardiography and an arterial blood flow is measured by means of photoplethysmography. The blood pressure is then determined from the time interval between a heartbeat and a pulse wave in the arteries triggered thereby and measured by the photoplethysmography. This is done by taking advantage of the fact that the blood pressure is correlated with the time span between the heartbeat and the resulting pulse wave in the arteries triggered thereby.

The time between a heartbeat and the resulting pulse wave in the arteries is also called pulse transit time.

International Patent Application Publication WO 89/08424 A1 and corresponding U.S. Pat. No. 5,237,997 relate to a method for the continuous measurement of blood pressure in humans. To determine one of the three blood pressure quantity (systolic, diastolic or mean blood pressure), the pulse transit time is measured continuously, making use of a proband-specific calibration curve which indicates the pulse transit time as a function of the blood pressure quantity used. To measure the pulse transit time, an ECG is recorded by means of two electrodes placed over the patient's heart and a sensor is attached to the earlobe with an ear clip. A small light source of the sensor shines through the earlobe and the transmission of the earlobe, which varies proportionally with the blood pressure, is measured by a photodiode. The temporal transmission curve shows the arrival of the pulse wave at the earlobe relative to the systole registered by the ECG signal. Thus, the pulse transit time is determined for the distance between the heart and the earlobe.

### SUMMARY OF THE INVENTION

It is an object of the present invention to provide a solution by which a reliable, accurate, fast and/or non-invasive, in particular cuff-free, medical examination, in particular blood pressure measurement, of animals such as dogs or cats is made possible and the examination or measurement is made as pleasant as possible for the animal.

The above object is solved by an examination apparatus, a method and a method of use according to the present invention as described herein.

The present invention in particular relates to an examination apparatus for the medical examination of an animal. The examination apparatus is in particular designed for the determination of a blood pressure, in particular also for the determination of a diastolic blood pressure.

Furthermore, the examination apparatus is preferably configured and/or suitable for the examination of animals having a paw, preferably animals from the superfamily of the Feloidea (cat-like) or Canoidea (dog-like), in particular animals from the family of the Felidae (cats) or Canidae (dogs), particularly preferably animals from the subfamily of the Felinae (small cats) or the tribe of the Canini (true dogs), in this tribe particularly animals of the genus Canis (wolf-like and jackal-like), particularly preferably domestic cats or domestic dogs.

In principle, however, the examination apparatus according to the invention is, alternatively or additionally, suitable for the medical examination, in particular blood pressure determination, of any animals, in particular humans.

The examination apparatus has a sensor device for the optical examination of an arterial blood flow of the animal. Preferably, the examination apparatus is designed for percutaneous and/or non-invasive examination of the blood flow of the animal. Particularly preferably, the sensor device and/or examination apparatus is designed for performing a photoplethysmography.

For the examination of an animal, it is preferably intended that a body part of the animal, in particular a paw, is positioned on or above the sensor device so that the arterial blood flow can be examined with the sensor device. Preferably, herein the body part or paw is not fixed relative to the sensor device and/or the body part or paw can be moved freely relative to the sensor device hereby. Hereby, the examination can be made very pleasant and stress-free for the animal. This is advantageous for a correct and/or meaningful result of a blood pressure determination, because it has been shown that under stress, e.g. caused by fixation of the animal or by manual manipulation on the animal, the blood pressure can change quickly and significantly. In this respect, it leads to a falsification of the result if the animal is under stress during the examination or during the blood pressure determination.

The sensor device has at least one emitter for emitting electromagnetic radiation and at least one detector for detecting the radiation emitted by the emitter. The electromagnetic radiation is preferably light including infrared light and/or ultraviolet light.

According to a first aspect, the sensor device has several emitters and several detectors arranged in a recurring or repeating structure, in particular in a periodic structure. This is conducive to a reliable and accurate examination, in particular blood pressure determination. In particular, hereby a larger area or region may be detectable or measurable by means of the sensor device, so that several, in particular simultaneous, measurements at different points of a paw are possible and/or there is a certain freedom in the placement of a paw over the sensor device. In addition, hereby a movement of the paw relative to the sensor device can be allowed or enabled during an examination. In this way, the examination can be made pleasant for the animal and thus stress-free. This is conducive to an accurate and reliable examination, in particular blood pressure measurement.

According to another aspect which can also be realized independently, the sensor device has a limiting device that defines a border of a sensing region of the sensor device so that the distance of the border of the sensing region from the sensor device is more than 0.5 mm and/or less than 5 mm. In this way, a reliable examination of the arterial blood flow is made possible and a minimum depth of penetration into the paw can be achieved and/or it can be avoided that the detector measures reflections from the outer face of the paw.

Preferably the sensor device has several emitters and several detectors. Here, it is preferred that the sensor device has at least four detectors and/or at least nine emitters. Particularly preferably, several, in particular at least or exactly four, emitters are assigned to each detector. This is conducive to a reliable and accurate examination, in particular blood pressure determination.

It is preferred that the emitters and detectors are arranged in a matrix with columns and rows. Here, the emitters and detectors are preferably arranged equidistantly. The matrix preferably has more than two columns and/or more than two rows. Particularly preferably, the emitters and detectors are arranged alternately in the columns and rows. In other words—with the exception of the emitters and detectors arranged at the edge of the matrix—in both the columns and the rows the emitters are arranged between two detectors in each case and the detectors are arranged between two emitters in each case. This is conducive to a reliable and accurate examination, in particular the determination of blood pressure.

The limiting device preferably limits an emission angle of the emitter and/or a detection angle of the detector to less than 90°, preferably about 60°. For this purpose, the limiting device can be designed as a barrier. However, it is also possible that the limiting device has an optical lens or is formed thereby, wherein a corresponding emission angle and/or detection angle is achieved by focusing or scattering by means of the lens.

The limiting device preferably has or is formed by a barrier to the radiation emitted by the emitter(s). The barrier is arranged between the emitter(s) and the detector(s) and in this way limits an emission region of the emitter(s) and/or a detection region of the detector(s) so that a sensing region of the sensor device is formed, the border of which is at a distance of more than 0.5 mm and/or less than 5 mm from the sensor device. In this way, it can be achieved that light scattered from a surface of the paw is switched off and/or blanked out and/or does at least essentially not reach the detector(s) and/or a minimum penetration depth of the radiation emitted by the emitters and detected by the detectors can be ensured.

Preferably, a height and/or width of the limiting device, a distance of the limiting device from the adjacent or associated emitter(s) and the adjacent or associated detector(s) and a distance between the emitter(s) and the detector are matched to each other in such a way that an emission region of the emitter and a detection region of the detector overlap in such a way that the distance of the border of the sensing region from the sensor device is more than 0.5 mm and/or less than 5 mm.

The examination apparatus preferably has one or several electrodes for recording a cardiogram, in particular an electrocardiogram. Preferably, at least one of the electrodes is arranged in such a way that a cardiogram can be recorded at a paw of the animal by means of the electrode and at the same time the optical examination can be carried out at this paw by means of the sensor device. This is conducive to an accurate and fast examination, in particular the determination of blood pressure. In addition, the examination can be made more pleasant for the animal and thus induces less stress for the animal, since no electrodes have to be fixed to the animal and/or the animal can move freely relative to the electrodes. This is conducive to an accurate and reliable examination, in particular blood pressure determination.

The sensor device preferably has a cover that is transparent to the radiation emitted by the emitter(s). Hereby, the sensor device can be protected from damage and/or contamination.

Particularly preferably, an electrode, in particular for recording a cardiogram, is preferably arranged on a side of the cover facing away from the emitter(s) and detector(s). This allows simultaneous recording of the cardiogram and optical examination by means of the sensor device on one or the same paw.

Here, it is particularly preferred that the electrode is arranged between and/or offset to the emitter(s) and the detector(s) in a projection perpendicular to the cover and/or opposite to the barrier. The electrode, acting as a mask, can form at least part of the barrier or be arranged in an area that is not covered or sensed by the emitter(s) and/or detector(s). Alternatively, or additionally, the electrode may be transparent for the radiation emitted by the emitter(s). This makes possible a simultaneous recording of a cardiogram and optical examination using the sensor device on the same paw. This simplifies the examination and makes it more pleasant for the animal, thus inducing less stress for the animal. This is conducive to accurate and reliable examination, in particular blood pressure measurement.

The sensor device preferably has more than 30, preferably more than 60 and/or less than 500, preferably less than 200, emitters. Alternatively, or additionally, the sensor device has more than 20, preferably more than 40, and/or less than 500, preferably less than 200, detectors. This is conducive to a reliable and accurate examination, in particular blood pressure determination. In particular, this increases the sensor area, making it easier to place the paw of the animal on the sensor device in such a way that the examination can be performed and/or the examination can be performed even if the paw is moved relative to the sensor device during the examination. In other words, the sensor device and/or examination apparatus is preferably designed to enable or permit movement of the animal during the examination and/or to enable a reliable and accurate examination, in particular blood pressure determination, and/or to reduce, avoid and/or compensate for movement artifacts. This makes the examination more pleasant for the animal and induces less stress for the animal. This is conducive to an accurate or reliable examination, in particular blood pressure measurement.

Preferably, an area density of the emitters, an area density of the detectors and/or a common area density of the emitters and the detectors is more than 0.5/cm², preferably more than 1/cm², in particular more than 2/cm², and/or less than 40/cm², preferably less than 20/cm², in particular less than 10/cm². This is conducive to a reliable and accurate determination of blood pressure.

It is preferred that the emitters are designed to emit radiation of the same wavelength and that the detectors are designed to detect at the same wavelength. It is in particular preferred that the emitters are identical in construction and/or that the detectors are identical in construction. This allows the different detectors or sensors to record comparable signals or signals of the same kind—preferably from different locations, in particular from locations that are offset to each other along the sensor device. In particular, this way the signals recorded by the different detectors and/or sensors contain basically the same or similar information. This is conducive to a reliable and accurate examination, in particular blood pressure determination, even when the animal under examination is moving. This makes the examination more pleasant for the animal and thus induces less stress for the animal. This is conducive to an accurate and reliable examination, in particular the determination of blood pressure.

Preferably the emitter(s) is/are designed to emit infrared radiation and/or radiation with a wavelength of more than 780 nm, preferably more than 900 nm, and/or less than 1400 nm, preferably less than 1100 nm, in particular about 940 nm and/or 1050 nm. This may make the examination, in particular the determination of blood pressure, very pleasant for the animal, since infrared radiation is not perceived. Furthermore, the use of infrared radiation has proven to be surprisingly advantageous for animals with heavily pigmented or dark paws or pads.

The examination apparatus is preferably at least essentially flat, mat-like and/or plate-like and/or in the form of a mat and/or plate. This has proven to be particularly advantageous for the examination of animals such as cats and dogs. In particular, it allows a cuff-free and non-invasive examination, in particular blood pressure determination. The examination can thus be made very pleasant and stress-free for the animal. This is conducive to an accurate and reliable examination, in particular the determination of blood pressure.

According to another aspect, which can also be realized independently, the examination apparatus is designed as a support for at least one paw of the animal, in particular as a support for the entire animal. Particularly preferably, the examination apparatus or support is designed in such a way that the animal, in particular a domestic cat or a domestic dog, can be completely positioned on the support during the examination and/or is movable freely relative to the support. Hereby, the examination can be made particularly pleasant and thus stress-free for the animal. This is advantageous for a correct and/or meaningful result of a blood pressure determination, because it has been shown that under stress, e.g. caused by fixation of the animal or by manual manipulation on the animal, the blood pressure can change quickly and significantly. Therefore, if the animal is under stress during the examination or during the blood pressure determination, the result is distorted.

The examination apparatus has a sensor device for the optical examination of an arterial blood flow of the animal. Preferably, the examination apparatus is designed for the percutaneous and/or non-invasive examination of the blood flow and/or animal. Particularly preferably, the sensor device and/or examination apparatus is designed for performing a photoplethysmography.

For the examination of an animal, it is preferably intended that a body part of the animal, in particular a paw, is positioned on or above the sensor device so that the arterial blood flow can be examined with the sensor device. Preferably, the body part or paw is not fixed relative to the sensor device and/or the body part or paw can be moved freely relative to the sensor device. Hereby, the examination can be made very pleasant and thus stress-free for the animal. This is advantageous for a correct and/or meaningful result of a blood pressure determination, because it has been shown that under stress, e.g. caused by fixation of the animal or by manual manipulation on the animal, the blood pressure can change quickly and significantly. In this respect, it leads to a falsification of the result if the animal is under stress during the examination or during the blood pressure determination.

Preferably, the sensor device is designed for examination with electromagnetic radiation in the infrared range. This has proven to be particularly advantageous, in particular for animals with heavily pigmented or dark paws or pads.

According to another aspect, which can also be realized independently, the examination apparatus has at least two, preferably three, detection elements for the detection of an activity of the animal's heart. The detection elements are preferably formed by electrodes for recording a cardiogram, in particular an electrocardiogram. This is conducive to a simple determination of the blood pressure. In principle, however, the detection elements can also be formed by microphones for recording a phonocardiogram (PPG) or the like.

According to another aspect, which can also be realized independently, the examination apparatus has at least one tissue electrode. This has proven to be advantageous in the examination of animals such as cats compared to the use of metallic electrodes. It has been shown that cats in particular often react irritated to metallic electrodes and that, in contrast, the use of tissue electrodes can make the examination with the examination apparatus more pleasant for cats and thus less stress is induced for the animal. This is conducive to an accurate and reliable examination, in particular blood pressure determination.

According to another aspect, which can also be realized independently, the examination apparatus has or forms a scale. Hereby, the accuracy of a determination of a blood pressure can be improved.

Preferably, a detector with one or more emitters forms one sensor each, so that the sensor device has several sensors. The sensors are designed for the in particular simultaneous recording of several curves comprising information about the arterial blood flow, in particular photoplethysmograms (PPGs). This is conducive to a fast, reliable and accurate determination of a blood pressure.

The electrodes are preferably arranged at a distance of more than 5 cm and/or less than 20 cm. In this way, the examination apparatus is particularly well adapted to dogs and/or cats, so that the examination is made as pleasant as possible for the dog or cat and can be performed quickly.

The examination apparatus preferably has a reference electrode or collection electrode and two further electrodes. This is advantageous for an accurate and reliable recording of a cardiogram.

The examination apparatus preferably has a rest surface. Preferably, an animal from the subfamily of the Felinae or from the family of the Canidae, in particular a domestic cat or a dog, can be completely placed on the rest surface. Preferably, the rest surface has a width of more than 20 cm, preferably more than 40 cm, and/or less than 80 cm, preferably less than 60 cm, and/or a length of more than 40 cm, preferably more than 60 cm, and/or less than 120 cm, preferably less than 80 cm. Hereby, the examination can be made particularly pleasant and thus stress-free for the animal. This is conducive to an accurate or reliable examination, in particular blood pressure determination.

The scale and/or the examination apparatus is preferably designed for body fat measurement. In particular, the examination apparatus is designed to determine the blood pressure of the animal taking into account the body fat measurement. The measurement of the body fat enables in particular a more exact determination of the blood pressure.

According to another aspect which can also be realized independently, the present invention concerns a method for medical examination, in particular determination of a blood pressure, of an animal having a paw, in particular an animal from the subfamily of the Felinae or the family of the Canidae, particularly preferably a domestic cat or a domestic dog, wherein the animal is positioned on an examination apparatus in such a way that a paw of the animal rests on a sensor device of the examination apparatus. By means of the sensor device, a curve comprising information about an arterial blood flow of the animal, in particular a photoplethysmogram, is then recorded. In this way, a medical examination, in particular the determination of blood pressure, can be made particularly pleasant and thus stress-free for the animal. This is achieved in particular by preferably not attaching or fixing any means for medical examination such as sensors, electrodes, clips or the like to the animal and by allowing the animal to move freely on or relative to the examination apparatus. This is conducive to an accurate and reliable examination, in particular blood pressure measurement.

According to a first aspect of the method, a reflective measurement with electromagnetic radiation in the infrared range is performed to record the curve. A reflective measurement has proven to be particularly advantageous because it only requires a paw to be placed on a sensor device and does not require the paw to be fixed or a device to be placed against a paw, as is the case with a cuff or clip. Hereby, the examination can be made particularly pleasant for the animal In a reflective measurement, an emitter and a detector are preferably located on the same side of the paw, wherein the light emitted by the emitter is reflected and/or scattered within the paw and thus reaches the detector. In principle, however, a transmissive measurement is also possible in which the emitter and the detector are located on opposite sides of the paw and the light transmitted through the paw is recorded with the detector. Furthermore, the use of infrared radiation has proven to be particularly advantageous for dogs and cats, as this radiation is not perceptible to the animals and thus the examination can be made particularly pleasant.

According to another, also independently realizable aspect of the method, a cardiogram, in particular an electrocardiogram, of the animal is recorded by means of the examination apparatus. This is conducive to a particularly accurate and reliable determination of blood pressure.

According to a further, also independently realizable aspect of the method, a signal is recorded by means of at least one tissue electrode. The use of a tissue electrode has proven to be particularly convenient for animals such as cats.

According to another aspect of the method, which can also be realized independently, the animal is weighed by means of the examination apparatus. Hereby, the accuracy in determining the blood pressure can be increased.

Preferably, a curve feature, in particular a pulse transit time, is determined by means of the curve and the blood pressure is determined on the basis of the curve feature or the pulse transit time by means of a preferably empirically determined correlation function.

The curve and the cardiogram are preferably recorded at the same time, in particular wherein the cardiogram is used to cut the curve into sections corresponding to heartbeats. This is conducive to an accurate determination of the pulse transit time and/or the blood pressure.

Preferably, a presence of the animal and/or a positioning of the animal on the examination apparatus is determined by means of the examination apparatus, in particular by evaluating signals measured with electrodes, the sensor device, a force sensor and/or the balance. For example, it can be determined with the sensor device whether and/or at which position a paw of the animal is positioned above the sensor device and/or whether the paw is positioned in such a way that the signals recorded by the sensor device contain information about an arterial blood flow of the animal. Alternatively, or additionally, it can be determined by means of electrodes, for example by a resistance measurement, whether the animal is correctly positioned, in particular whether the electrodes are contacted, for example with a paw. Finally, the weight measured by the scale also provides information about whether the animal has already been positioned on the examination apparatus and/or whether the animal completely resides on the examination apparatus.

By means of the scale and/or the examination apparatus, a body fat measurement is preferably performed. Particularly preferably, a blood pressure of the animal is determined under consideration of the body fat measurement and preferably under simultaneous consideration of the weight of the animal measured with the scale. The consideration of the body fat leads in particular to a more exact and more reliable determination of the blood pressure.

According to a further aspect, the present invention relates to a use of the examination apparatus for medical examination, in particular determination of a blood pressure, of animals having a paw, in particular animals from the subfamily of the Felinae or the family of the Canidae, particularly preferably domestic cats or domestic dogs.

As a result, the present invention makes it possible to measure blood pressure in animals, in particular also in animals which, according to experience, have a high urge to move and/or a low stress tolerance with regard to manipulation of the animal's body, as is the case in particular with domestic dogs and domestic cats.

Here, in the past, a blood pressure measurement was always associated with considerable stress for the animal. The present invention solves this problem by a complete departure from known approaches in which animals are fixed and/or sensor technology is fixed to animals. The present invention provides a remedy in an unpredictable and surprising way by combining measures which—instead of requiring a restriction of movement—do not restrict the freedom of movement at least essentially. Instead of fixing the animal, measurement problems that may be caused by a possible movement of the animal during the examination are technically solved. In particular, so-called movement artifacts, i.e. measurement inaccuracies and measurement errors caused by movement, are eliminated and/or compensated.

In order to achieve this goal, different measures are described and/or applied, which can be realized individually, but interdigitate with each other and thus enable a particularly reliable and equally low-stress blood pressure determination in a synergistic way.

So on the one hand it is preferably intended that the position of the animal, in particular thus the position of the paw, is not strictly given. Instead, several sensors are used and the sensor that is suitable for a measurement can be selected.

This is preferably combined with further measures, each of which can be implemented individually and combined in a particularly advantageous way, in order to preferably ultimately determine a curve feature from the measured curve(s), and in particular to determine a blood pressure on the basis of the curve feature.

Particularly advantageous and the basis of some of the further measures is the subdivision or cutting of signals or curves into curve sections on the basis of the simultaneously determined cardiogram. Another basis of most of the proposed measures is the averaging between the curve sections.

In addition, there is in particular the selection of suitable curve sections and/or the selection from several alternative results determined for the curve feature and/or filter measures and/or statistical methods. In particular, these and further measures described in detail lead to the fact that a simple placing of a paw or paws on or at the sensor device and/or putting the animal on the examination apparatus is sufficient to achieve a meaningful determination of the curve feature and a reliable determination of the blood pressure therefrom. This seemed to be impossible in this form before.

An “animal” in the sense of the present invention is preferably a vertebrate, in particular a mammal, particularly preferably a land mammal. In particular, the term “animal” within the meaning of the present invention also includes humans. Preferably, the animal to be examined has a paw. Preferably, the animal to be examined is an animal from the superfamily of the Feloidea (cat-like) or Canoidea (dog-like), in particular an animal from the family of the Felidae (cats) or Canidae (dogs), in particular preferred is an animal from the subfamily of the Felinae (small cats) or the tribe of the Canini (true dogs), in this tribe in particular an animal of the genus Canis (wolf-like and jackal-like), particularly preferred a domestic cat or a domestic dog.

An “emitter” in the sense of the present invention is preferably a structure which is emits or is designed to emit electromagnetic radiation, in particular in the optical and/or infrared range. Preferably, an emitter is formed by a light-emitting diode, a laser diode, or generally a light-generating element. However, an emitter can also be formed by the end of an optical fiber at which light guided by the optical fiber exits—at least as far as a position of the emitter is concerned. Depending on the point of view, the combination of the light guide with its associated light source is then the emitter. In principle, the term “emitter” in the sense of the present invention is therefore preferably to be understood broadly.

A “detector” in the sense of the present invention is preferably a structure which is designed to detect electromagnetic radiation, in particular in the optical and/or infrared range. Preferably, a detector is formed by a photodiode. In principle, however, a detector can also be formed by another structure which is designed for the detection of electromagnetic radiation emitted in particular by the emitter, for example a photocathode, a photocell, a CCD sensor or the like. The detector may also have a light guide with one end where light guided by the light guide can enter. In this case, the end of the light guide is the detector, at least as far as a position of the detector is concerned.

An “emission region” of an emitter in the sense of the present invention is preferably a region into which radiation emitted by the emitter reaches or can reach. Preferably, an emitter emits radiation in a certain direction, for example in a certain angular range. The emission region is therefore preferably defined or limited by one or more emission angles. The emission region can be essentially conical.

A “detection region” of a detector in the sense of the present invention is preferably a region from which radiation reaches or can reach the detector. A detection region is preferably defined or limited by one or more detection angles. The detection region can be essentially conical.

A “sensor” in the sense of the present invention is preferably a combination of at least one emitter with at least one detector. In particular, a detector with one or more emitters forms a sensor in the sense of the present invention. A sensor preferably comprises exactly one detector and at least one emitter. The emitter is designed to emit electromagnetic radiation with a wavelength at which the detector is sensitive and/or can detect this electromagnetic radiation.

A “sensor region” of a sensor in the sense of the present invention is preferably a region which is detectable/sensable by means of the sensor or in which a measurement can be made by means of a sensor. In particular, a sensor region is a region in which the emission region of an emitter and the detection region of a detector of the sensor overlap. A sensor region can be formed by a continuous region or by several disjunctive or separated regions.

A “sensor device” in the sense of the present invention is preferably a device having one or more sensors. In particular, a sensor device is a device for optical examination of a body part of an animal. The sensor device is in particular designed for performing a photoplethysmography.

A “sensing region” of the sensor device in the sense of the present invention is preferably a region which is detectable/sensable by means of the sensor device and/or the emitters and/or the detectors. The sensing region is in particular a region in which an emission region of an emitter and a detection region of a detector overlap. Preferably, the sensing region is formed by one or more emission regions and one or more detection regions that overlap. The sensing region can be connected or can be formed by several separate regions. In particular, the sensing region can be formed by one or more overlapping regions of essentially conical emission and detection regions.

A “periodic” arrangement of emitters and/or detectors in the sense of the present invention is preferably an arrangement in which the emitters and/or detectors are arranged in a structure which is repeated at least substantially equal intervals. Such periodicity can be present in one or more directions, which are in particular orthogonal to each other.

An “optical examination” in the sense of the present invention is preferably an examination in which a body part of an animal is irradiated with electromagnetic radiation in the optical range and/or range visible to humans and/or in the infrared range, in particular with a wavelength between 380 nm and 1400 nm, and in which the radiation reflected and/or scattered by the body part and/or radiation transmitted through the body part is measured by means of a detector. The optical examination is preferably a reflectometric examination. Conclusions can then be drawn from the reflected, scattered and/or transmitted radiation, for example with regard to the arterial blood flow. In particular, electromagnetic radiation of a defined wavelength or a defined wavelength range is used in an optical examination. Particularly preferably, an optical examination is a non-invasive and/or percutaneous examination of the inside of the body.

A “photoplethysmography” in the sense of the present invention is a method for optical examination of an arterial blood flow of an animal. In particular, a photoplethysmography is a method for non-invasive optical examination in which a body part of an animal is irradiated with electromagnetic radiation, in particular in the range visible to humans and/or the infrared range, and the radiation scattered and/or (in particular diffusely) reflected and/or transmitted by the body part is measured by means of a detector. The reflection and/or scattering and/or transmission, in particular the proportion of the electromagnetic radiation reflected or transmitted in the direction of the detector, depends, among other things, on the arterial blood flow, in particular the volume of the arterial blood and/or the oxygen saturation of the arterial blood. Preferably, the variation of the arterial blood flow and/or the change in volume and/or the change in oxygen saturation of the arterial blood changes the signal measured by the detector, so that variations in the measured signal and/or the course of the measured signal allow conclusions to be drawn about the arterial blood flow. Accordingly, pulse oximetry is also an (extended) photoplethysmography in the sense of the present invention.

In the sense of the present invention, a pulse oximetry comprises at least one photoplethysmography. In a pulse oximetry, the oxygen content in the blood is determined, wherein two photoplethysmographies are carried out, in particular simultaneously, to determine the oxygen content, wherein different wavelengths are used for these two photoplethysmographies. From the different absorption rates at the two wavelengths, the oxygen saturation of the blood can then be determined.

A “photoplethysmogram” (PPG) in the sense of the present invention is in particular the curve recorded or measured during the performance of a photoplethysmography.

However, also known from the state of the art are optical examinations, for example to determine the oxygen content in the blood, that do not represent or include photoplethysmography. In particular, the methods of cerebral oximetry and tissue oximetry do not include photoplethysmography. These methods are also not suitable for examination of the arterial blood flow, in particular due to the wavelengths of the electromagnetic radiation used.

A “cardiogram” in the sense of the present invention is preferably a curve representing the activity of the heart of the animal. Particularly preferably, the cardiogram is recorded electrically, in particular by means of electrodes which are brought into contact with the skin of the animal, and/or is an electrocardiogram. In principle, however, other methods for recording a cardiogram are also conceivable, for example an impedance cardiogram or an acoustic recording, so that the cardiogram is a phonocardiogram.

A “detection element” in the sense of the present invention is preferably an element for detecting an activity of the heart of the animal. A detection element is in particular suitable or designed for recording a cardiogram. A detection element is preferably formed by an electrode. However, the detection element may also be formed by a microphone or other sound sensor or the like or have this/these.

An “arterial blood flow” in the sense of the present invention is preferably the flow of blood through the arteries. Arteries are in particular blood vessels that lead the blood away from the heart. In particular, the arterial blood flow is a blood flow of the animal to be examined.

A “blood pressure” in the sense of the present invention is preferably a pressure (force per area) of the blood in a blood vessel, in particular a blood vessel of the animal to be examined. The blood vessel is preferably an artery. Preferably, the blood pressure is a blood pressure in the larger arteries. The blood pressure can be a systolic, diastolic and/or mean blood pressure. In particular, it has been surprisingly shown in the context of the present invention that the proposed method and/or examination apparatus can also be used for the determination of a diastolic blood pressure. This is, however, not mandatory.

A “curve” in the sense of the present invention is preferably the time course of a signal measured by means of a detector or sensor. The term “curve” also includes data-technical equivalents such as individual data points, which (together) represent or correspond to the course. A curve is preferably a temporal course over several heartbeats.

A “curve section” in the sense of the present invention is preferably a section or part of a curve, i.e. in particular also a time course of a signal measured by a detector or sensor. In particular, a curve section is a section of a curve corresponding to a heartbeat, in particular beginning at the time of a heartbeat and preferably ending at the time of a subsequent heartbeat.

A “curve comprising information about an arterial blood flow” in the sense of the present invention is in particular a curve which allows conclusions to be drawn about the arterial blood flow, in particular the arrival of a pulse wave, the change in the blood volume in the arteries, the change in the oxygen saturation of the blood in the arteries or the like. A photoplethysmogram (PPG) is a particularly preferred example of a curve comprising information about arterial blood flow.

A “curve feature” in the sense of the present invention is preferably a feature of a curve and/or a section of a curve, which in particular comprises information about an arterial blood flow. The curve feature is preferably a feature which is related to a pulse transit time and/or a blood pressure, and/or is correlated with a pulse transit time and/or a blood pressure. In particular, a curve feature is a feature by means of which the blood pressure can be determined. The curve feature is particularly preferably a feature of the curve and/or the curve section that corresponds to a course and/or a form of the curve and/or the curve section and/or contains information about a form of the curve and/or the curve section. For example, the curve feature can be a position of an (absolute) extremum, a distance between (absolute) extrema, a position or an absolute value of a (maximum) slope, a distance between extrema and/or zero points of the first and/or second derivative of the curve or a feature of a Fourier transform of the curve.

Particularly preferably, the curve feature corresponds to a pulse transit time.

A “pulse transit time” in the sense of the present invention is preferably the time required by a pulse wave to travel a distance in the vascular system. Herein, the pressure wave which passes through the arteries—starting from the heart due to a heartbeat—is denoted as pulse wave. The velocity of this pressure wave is in particular higher than the flow velocity with which the blood flows through the arteries. The pulse transit time is often abbreviated as “PTT”. In particular, in the present invention, the term pulse transit time comprises the time between a heartbeat and the arrival of the pulse wave caused by this heartbeat at a specific location of an artery, i.e. the time required for the pulse wave to travel the distance from the heart to the location of the artery. Preferably, however, the term pulse transit time also includes the time distance between the arrival of the pulse wave at a first location and a second location.

A “pulse wave velocity” in the sense of the present invention is preferably the quotient between the distance travelled by the pulse wave and the pulse transit time required by the pulse wave to travel this distance. The pulse wave velocity is often abbreviated as “PWV”.

A “percutaneous” examination in the sense of the present invention is preferably an examination through the skin. In an optical percutaneous examination, the interior of the body is preferably irradiated through the skin with electromagnetic radiation in the (for humans) optically visible range and/or infrared range and scattered, transmitted and/or reflected portions thereof are detected.

A “non-invasive” examination within the meaning of the present invention is preferably an examination in which the animal to be examined is not damaged or injured.

The above-mentioned aspects and features as well as further aspects and features resulting from the claims and the following description can be realized independently from each other and in different combinations.

Further advantages, features, properties and aspects of the present invention will become apparent from the following description of preferred embodiments with reference to the accompanying drawings.

### BRIEF DESCRIPTION OF THE DRAWINGS

FIG. 1 is a schematic top view of an examination apparatus according to the invention;

FIG. 2 is a schematic perspective view of an examination apparatus according to the invention with an animal placed thereon;

FIG. 3 is a schematic top view of a sensor device according to a first embodiment;

FIG. 4 is a schematic top view of a sensor device according to a second embodiment;

FIG. 5 is a schematic sectional view through the sensor device;

FIG. 6 is a schematic exploded view of the sensor device with an electrode arranged thereon;

FIG. 7 is a schematic sectional view of the sensor device with a paw placed thereon;

FIG. 8 is a schematic, block diagram-like representation of the examination apparatus; and

FIG. 9 is a schematic representation of a cardiogram and a curve comprising information about arterial blood flow.

### DETAILED DESCRIPTION OF THE INVENTION

In the partly not true to scale, only schematic figures, the same reference signs are used for identical or similar parts, wherein corresponding or comparable characteristics and advantages can be achieved, even if a repeated description is omitted.

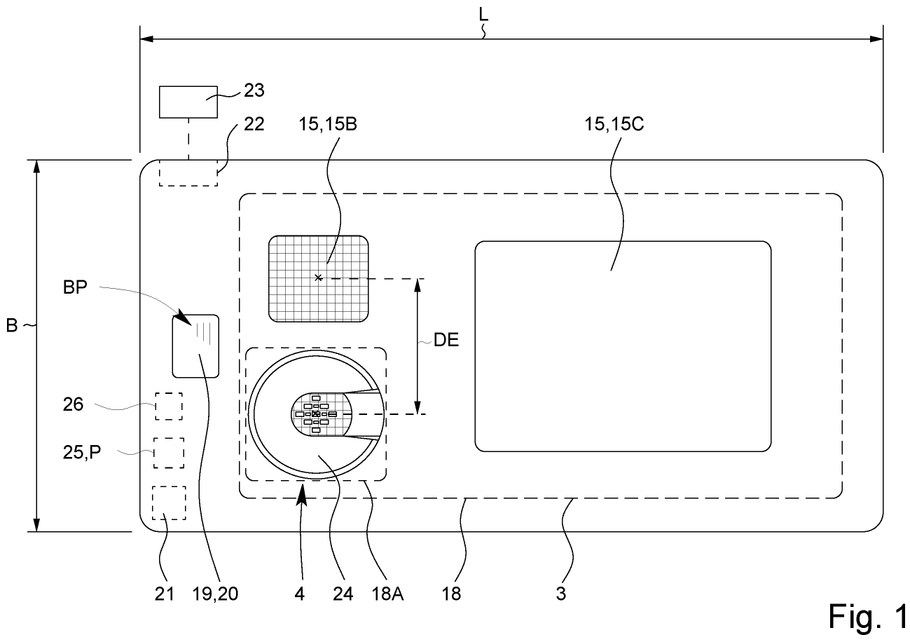

FIG. 1 shows an examination apparatus 1 that is preferably designed for medical examination, in particular for determining a blood pressure BP, of an animal T, in particular an animal T having a paw 2, preferably an animal T from the subfamily of the Felinae, particularly preferably a domestic cat.

In principle, however, the examination apparatus 1 is suitable for the medical examination of any animal T, in particular humans, in particular those in which a blood pressure BP can be determined. For examination using the examination apparatus 1, it is particularly advantageous if the animal T has a paw or the like.

However, the examination apparatus 1 may also be designed and/or suitable for the medical examination, in particular for the determination of blood pressure BP, of other animals T, in particular domestic animals, such as dogs, mice, rats, rabbits, guinea pigs or the like and/or specially adapted for the examination of these animals T.

The blood pressure BP can be a systolic, diastolic and/or mean blood pressure BP. In particular, it has been surprisingly shown in the context of the present invention that the proposed method and/or examination apparatus can also be used for the determination of a diastolic blood pressure BP. This is, however, not mandatory.

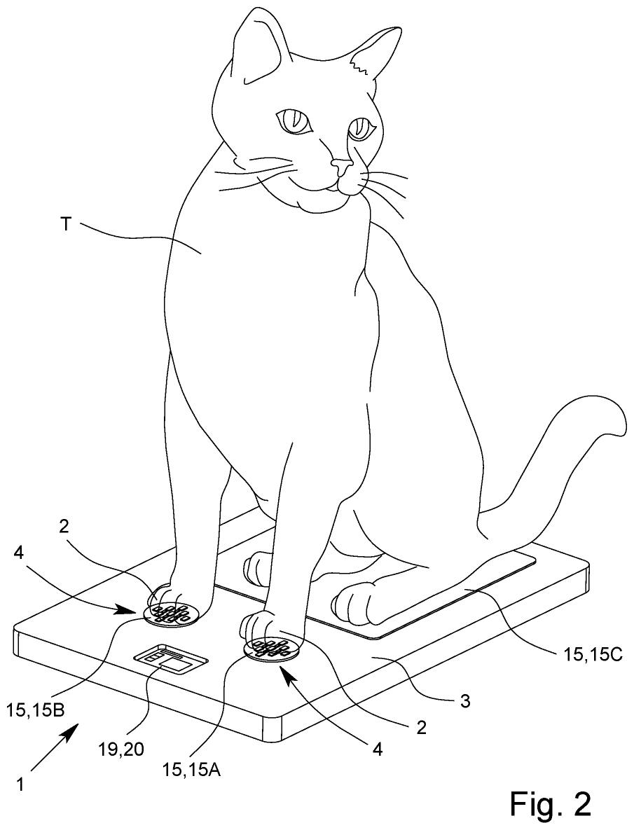

In FIG. 2, an examination apparatus 1 according to the invention is shown in a schematic perspective view with an animal T arranged on it. Preferably, the examination apparatus 1 is designed as a support for at least one paw 2 or any other part of the body, in particular a part similar to a paw, for example a hand or a finger, of the animal T.

Particularly preferably, the examination apparatus 1 and/or support is designed in such a way that the animal T to be examined can be completely placed and/or positioned on the examination apparatus 1 and/or support, in particular thus all legs of the animal T can be positioned on the examination apparatus 1. However, this is not mandatory. In principle, it is also possible that the examination apparatus 1 is designed so that only one or two paws 2 can be placed or positioned on the examination apparatus 1.

The examination apparatus 1 is preferably designed as mat or plate or mat-like or plate-like or in the form of a mat or plate. In particular, a plate or mat is understood to be a device whose width and length exceed the height by a multiple. A plate is preferably understood to be an at least substantially rigid apparatus. A mat is preferably understood to be an at least partially flexible apparatus. For example, if the examination apparatus 1 is designed as a mat, it may be at least partially rollable and/or foldable.

Preferably, the examination apparatus 1 has a rest surface 3. The animal T, in particular a domestic dog, a domestic cat or another animal T of comparable or smaller size, can be, preferably completely, placed on the rest surface 3.

Preferably, the examination apparatus 1 and/or rest surface 3 is at least essentially flat and/or planar.

Preferably, the examination apparatus 1 has the rest surface 3 on one upper side and/or the rest surface 3 is formed by an upper side of the examination apparatus 1 or a part thereof.

The rest surface 3 is or forms in its position of use, in particular during the examination, preferably an at least substantially horizontal surface. The position of use is a preferred position of the examination apparatus 1, in which the animal T can be placed on the examination apparatus 1 for examination. The position of use is in particular shown in FIG. 2.

The examination apparatus 1 and/or rest surface 3 preferably has a width B of more than 20 cm, preferably more than 40 cm, and/or less than 80 cm, preferably less than 60 cm.

The examination apparatus 1 and/or rest surface 3 preferably has a length L of more than 40 cm, preferably more than 60 cm, and/or less than 120 cm, preferably less than 80 cm. In principle, a different width B and/or a different length L of the examination apparatus 1 and/or rest surface 3 are also conceivable.

It is preferably intended that during the examination the examination apparatus 1 contacts the paw 2 and/or the body part only on one side, and/or rests or is arranged only on one side. The examination apparatus 1 is therefore preferably designed for one-sided contact with the animal T and/or its paw 2.

The examination apparatus 1 is preferably free of fixing means and/or fastening means. Preferably, the examination apparatus 1 is not designed to clasp the paw 2. Preferably, the examination apparatus 1 does neither have a clip for attachment to the paw 2 nor a cuff for application to the paw 2 or other fixing means or fastening means for attaching, fixing or fastening an examination means such as a sensor or an electrode to the animal T. In contrast, it is preferred that the examination apparatus 1 has a contact and rest surface 3, by which the examination is made possible when the paw 2 or body part is put on or placed on the device.

The design of the examination apparatus 1 as a support and/or with a rest surface 3 for the animal T makes the examination particularly pleasant and thus stress-free for the animal T. Preferably, it is not intended that the animal T is fixed to the examination apparatus 1 for examination or that a part of the examination apparatus 1, such as a sensor or the like, is attached or fixed to the animal T. It has been shown that such a method causes stress in an animal T, so that the examination would be unpleasant for the animal T and, in addition, the blood pressure BP would be influenced by the stress. In contrast, by designing the examination apparatus 1 according to the invention, the examination can be made very pleasant and stress-free for the animal T.

Preferably, the examination apparatus 1 or rest surface 3 is designed in such a way that the animal T can move freely on the examination apparatus 1 and/or rest surface 3.

By the design of the examination apparatus 1 described in more detail below, in particular the design and/or arrangement of the sensor device 4 and/or the electrodes 15, it is accomplished that an examination of the animal T, in particular a reliable and/or accurate blood pressure determination, is made possible while avoiding fixation of the animal T or can be made without fixation of the animal T and/or can be made or is made possible when the animal T moves during the examination by means of the examination apparatus 1.

The examination apparatus 1 preferably has a sensor device 4. The sensor device 4 is designed for the optical examination of an arterial blood flow BF of the animal T, in particular for recording a curve K that contains information about an arterial blood flow BF of the animal T. In particular, the sensor device 4 is designed to perform a photoplethysmography and/or to record a photoplethysmogram (PPG).

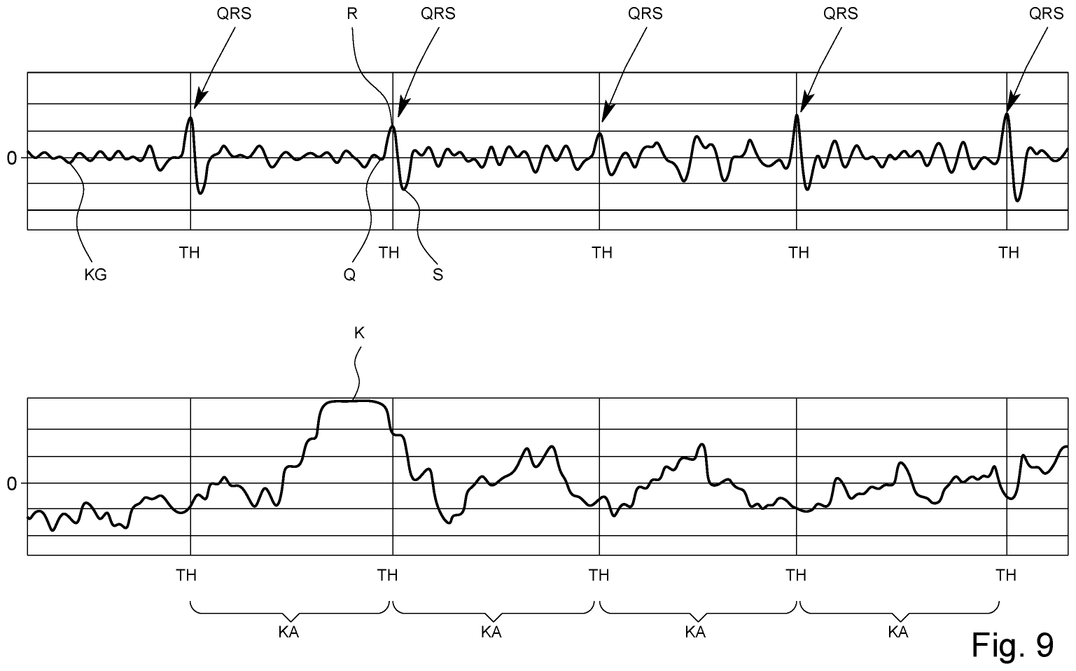

A curve K comprising information about the arterial blood flow BF is shown as an example in FIG. 9 and will be explained in more detail later.

The sensor device 4 and/or examination apparatus 1 is preferably designed to enable or allow movement of the animal T during the examination and/or to enable a reliable and accurate examination, in particular blood pressure determination, and/or to reduce, avoid and/or compensate for movement artifacts.

The examination apparatus 1 has the sensor device 4 preferably in the area of the rest surface 3. Thus, an examination with the sensor device 4 can be performed when the paw 2 or the body part is placed on the surface.

The sensor device 4 is preferably arranged at the examination apparatus 1 or integrated into the examination apparatus 1 in such a way that a paw 2 of the animal T can be positioned at, above and/or in the immediate vicinity of the sensor device 4, in particular if the animal T is located on the examination apparatus 1 and/or rest surface 3. In the example shown in FIG. 1, the sensor device 4 is positioned in such a way that the left forepaw 2 of the animal T can be positioned above the sensor device 4 without any problems and in a position that is pleasant and/or natural for the animal T. However, the sensor device 4 can also be provided at another position.

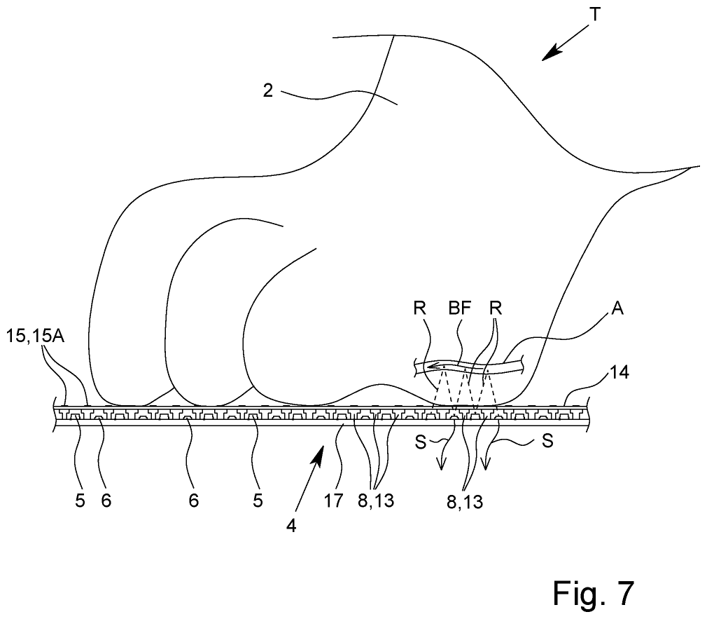

FIGS. 2 and 7 show, by way of example, the positioning of a paw 2 during an examination by means of the sensor device 4. For the examination by means of the sensor device 4, the paw 2 is preferably positioned in such a way that one or preferably several pads of the paw 2 contact the sensor device 4, in particular a cover 14 and/or electrode 15.

The examination apparatus 1 may also have several, in particular two, sensor devices 4, for example a sensor device 4 for the left forepaw 2 and a sensor device 4 for the right forepaw 2 of an animal T to be examined In this case, the sensor devices 4 are preferably of a similar or identical design. This is in particular shown in FIG. 2.

The sensor device 4 is preferably designed for a reflective measurement of an arterial blood flow BF.

The sensor device 4 has at least one emitter 5 for emitting electromagnetic radiation R—in particular light including ultraviolet light and/or infrared light—and at least one detector 6 for detecting electromagnetic radiation R, preferably emitted by the emitter 6—in particular light including ultraviolet light and/or infrared light.

The emitter 5 is preferably designed as a light emitting diode or laser diode.

The detector 6 is preferably designed as a photodiode.

Preferably, the emitters 5 can be activated and/or deactivated and/or switched on and/or off separately, in particular by means of MOSFETs assigned to the emitters 5.

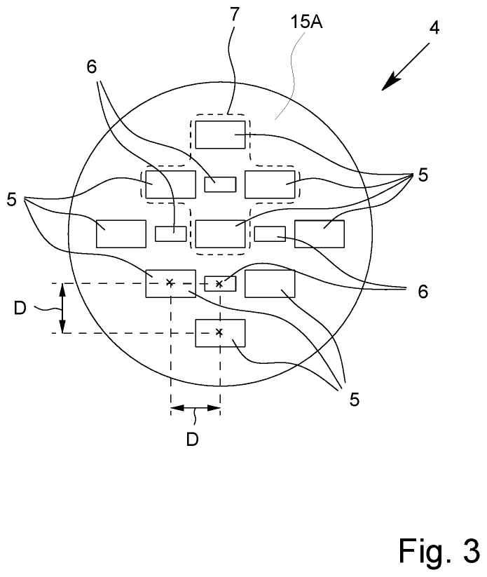

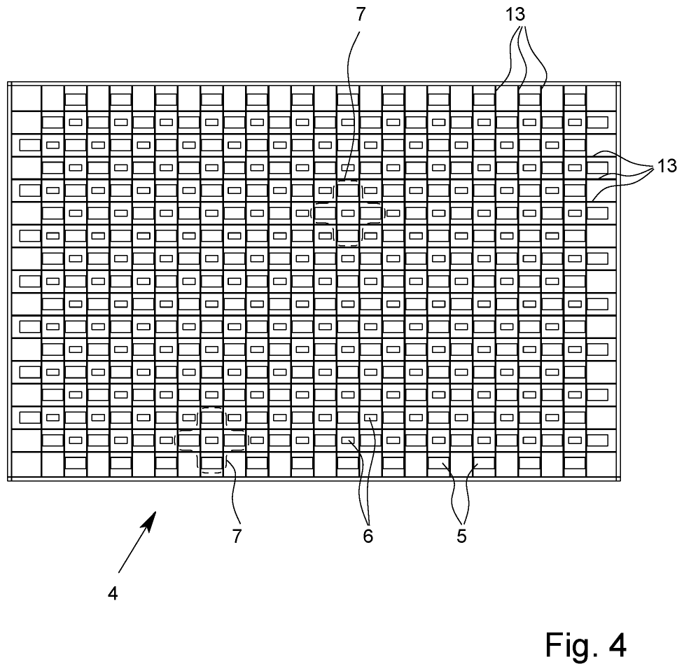

FIGS. 3 and 4 show an example of a schematic top view of a sensor device 4 in different embodiments. The sensor devices 4 according to FIGS. 3 and 4 are basically the same or similar in design and differ primarily only in the number of emitters 5 and detectors 6.

Preferably, the sensor device 4 has several emitters 5 and several detectors 6. In principle, however, it is also possible that the sensor device 4 has exactly one emitter 5 and exactly one detector 6 or exactly one emitter 5 and several detectors 6 or several emitters 5 and exactly one detector 6.

Preferably, however, the sensor device 4 has at least nine, in the example shown in FIGS. 1 and 3 exactly nine, emitters 5 and/or at least four, in the example shown in FIGS. 1 and 3, and exactly four detectors 6.

The emitters 5 and detectors 6 are preferably arranged in a common plane.

The emitters 5 and detectors 6 are preferably arranged in a recurring and/or repeating structure. Particularly preferably, the emitters 5 and detectors 6 are arranged periodically or in a periodic structure.

Preferably, the emitters 5 and the detectors 6 are arranged in the form of a matrix or in a matrix or an array with or in (virtual) columns and rows. Preferably, the matrix or array has more than two columns and/or more than two rows.

In other words, the emitters 5 and detectors 6 are preferably arranged in one or more, especially rectilinear, rows. Preferably, the emitters 5 and detectors 6 form several parallel rows and several rows running transversely, in particular perpendicularly, to each other, in particular where the rows form columns and rows of an (imaginary) matrix or (imaginary) array.

In other words, the emitters 5 and detectors 6 are preferably arranged in, in particular, a uniform grid.

The emitters 5 and detectors 6 are preferably arranged alternately. Preferably, the emitters 5 and detectors 6 form one or more in particular rectilinear rows, with emitters 5 and detectors 6 alternating in each row. The rows can also be curved and/or emulate an organic shape, such as that of a paw 2.

Particularly preferably, the emitters 5 and detectors 6 are alternately arranged in the columns as well as in the rows of the (imaginary) matrix.

Preferably,—as the case may be with the exception of the emitters 5 and/or detectors 6, which are the outermost and/or arranged at the edge of the sensor device 4 and/or rows and/or matrix—the detectors 6 are each (directly) surrounded by several emitters 5 and/or the emitters 5 are each (directly) surrounded by several detectors 6.

Particularly preferably, several emitters 5 are assigned to each detector 6 or vice versa. This allows preferably the multiple use of emitters 5 and/or detectors 6.

An emitter 5 and detector 6 are in particular assigned to each other if the emitter 5 and the detector 6 are arranged in such a way that the radiation R emitted by the emitter 5, in particular after scattering or reflection in a paw 2, reaches or can reach the detector 6. Particularly preferably, those emitters 5 are assigned to a detector 6 that have the smallest distance D to this detector 6 and/or are (directly) adjacent to this detector 6. Analogously, in particular those detectors 6 are assigned to an emitter 5 that have the smallest distance D to this emitter 5 and/or are (directly) adjacent to this emitter 5.

The distance D between an emitter 5 and a detector 6 is understood in particular as the distance between a center point or geometric center of the emitter 5 or its emission surface and a center point or geometric center of the detector 6 or its detection surface. Preferably, the emitters 5 and detectors 6 are formed by components of different sizes and/or rectangular components, as also indicated by the differently sized rectangles in FIGS. 1 to 4, wherein the emitters 5 and detectors 6 are arranged in such a way that the center points or geometric centers of gravity of these components, indicated by points in FIG. 3, have the same distance D from each other.

Preferably, the emitters 5 assigned to a detector 6 have the same distance D to the detector 6. Analogously, this also applies to the detectors 6 that are assigned to an emitter 5.

In the illustration example, exactly four emitters 5 are assigned to each detector 6 and/or exactly four detectors 6 are assigned to each emitter 5. The emitters 5 assigned to a detector 6 are preferably arranged symmetrically around the detector 6 and/or at equal distances D from the detector 6 and/or vice versa.

Preferably the emitters 5 and detectors 6 are arranged equidistant or at equal distances D from each other. In other words, a detector 6 has the same distance D to the two adjacent emitters 5 in the row in each case and/or to the four adjacent emitters 5 in the matrix in each case.

The distance D between emitters 5 and detectors 6 that are arranged directly adjacent to another, in particular in a column or row, is preferably more than 1 mm, in particular more than 2 mm, particularly preferably more than 4 mm, and/or less than 20 mm, in particular less than 15 mm, particularly preferably less than 10 mm, very particularly preferably between 5 mm and 7 mm.

Preferably, the emitters 5 of the sensor device 4 are of the same design or kind. Particularly preferably, the emitters 5 of the sensor device 4 are identical in construction and/or designed for emission at the same wavelength or in the same wavelength range.

Preferably, the detectors 6 of the sensor device 4 are of the same design or kind. Particularly preferably, the detectors 6 are identical in construction and/or designed for detection at the same radiation R or wavelength, in particular emitted by the emitters 5.

The sensor device 4 is preferably designed for examination with electromagnetic radiation R in the infrared range. Particularly preferably, the emitters 5 are designed for emission of infrared radiation and/or the detectors 6 are designed for detection of infrared radiation.

Infrared radiation is in particular electromagnetic radiation R with a wavelength between 780 nm and 1400 nm.

Preferably, the emitters 5 are designed for the emission of electromagnetic radiation R with a wavelength of more than 900 nm and/or less than 1200 nm or 1100 nm. Particularly preferably, the emitters 5 are designed for the emission of electromagnetic radiation R with a wavelength of more than 920 nm and/or less than 960 nm, in particular (approximately) 940 nm. Alternatively, or additionally, however, it is also possible that the emitters 5 or a subset of the emitters 5 is/are designed to emit electromagnetic radiation R with a wavelength of more than 1030 nm and/or less than 1070 nm, in particular (approximately) 1050 nm.

The detectors 6 are preferably designed to detect the radiation R emitted by the emitters 5.

Preferably, the sensor device 4 has at least one, preferably several, sensors 7. A sensor 7 has at least one emitter 5 and at least one detector 6 or is formed hereby. Particularly preferably, a sensor 7 has exactly one detector 6 and several emitters 5, in the example shown in FIG. 3 and FIG. 4 exactly four emitters 5.

Preferably, the emitters 5 of a sensor 7 are arranged symmetrically around the detector 6 of the sensor 7 and/or the emitters 5 of the sensor 7 have the same distance D to the detector 6 of the sensor 7.

In particular, the sensor device 4 has several sensors 7 which are of the same type or kind, in particular identical in construction. Particularly preferably, all sensors 7 of the sensor device 4 are identical. Here, however, other solutions are also possible. For example, the sensor device 4 could have two or more different types of sensors 7, wherein the sensor device 4 has several sensors 7 of each type. The different types of sensors 7 could differ for example in the number of emitters 5 and/or detectors 6, in the wavelength of the radiation R emitted by the emitters 5, in the distance of the emitters 5 from the detectors 6 or the like.

In the illustration example shown in FIG. 3, the sensor device 4 has exactly four sensors 7, one of the four sensors 7 being indicated by the dotted line in FIG. 2. Also in FIG. 4 some sensors 7 are indicated by dashed lines.

Preferably, an emitter 5 is assigned to several sensors 7 and/or the emitters 5 each form a part of several sensors 7 (apart from emitters 5, which are arranged at the outermost edge of the sensor device 4). In particular, each emitter 5 is assigned to the adjacent detectors 6 in the row or column and/or to the detectors 6 with the smallest distance D. In the illustration example, the emitters 5—apart from the emitters 5 arranged at the edge—are assigned to four detectors 6 each.

In the embodiment shown, several emitters 5 are assigned to each detector 6, wherein these emitters 5—except for the outermost emitters 5 or emitters 5 arranged at the edge —are, in turn, each assigned to several detectors 6. Hereby, several sensors 7, in particular of the same kind or type, are formed, wherein the emitters 5—except for the outermost emitters 5 or emitters 5 arranged at the edge—are each part of several sensors 7. In the example shown in FIG. 3, the emitter 5 arranged in the center of the sensor device 4 is assigned to each of the four detectors 6. The emitters 5 located in FIG. 3 at the very top, very bottom, very left and very right are assigned to only one detector 6 each. The remaining four emitters 5 in FIG. 3 are assigned to two detectors 6 each. In this way, four sensors 7, in particular of the same kind or type, are formed in FIG. 3.

While FIG. 3 shows the basic design of the sensor device 4 or the basic arrangement of the emitters 5, detectors 6 and/or sensors 7, the sensor device 4 preferably has a considerably larger number of emitters 5, detectors 6 and/or sensors 7, as shown in FIG. 4 as an example. In this way a large sensor area can be realized, so that the exact positioning of a paw 2 for examination and/or blood pressure determination is not or less decisive, but a larger area can be examined by means of the sensor device 4. This makes it possible that the paw 2 of the animal T does not have to be fixed, so that the stress during the examination is reduced for the animal T and a faster, more accurate, more reliable and for the animal T as pleasant as possible examination, in particular blood pressure determination, can be realized.

The sensor device 4 preferably has more than 30, in particular more than 60, and/or less than 500, preferably less than 200, more preferred less than 100, in particular less than 100, particularly preferably about 80, emitters 5.

Preferably, the sensor device 4 has more than 20, preferably more than 40, and/or less than 500, preferably less than 200, in particular less than 100, particularly preferably about 60, detectors 6.

Preferably, the number of sensors 7 corresponds to the number of detectors 6, since preferably a detector 6 with several emitters 5 forms a sensor 7. However, if an emitter 5 with several detectors 6 forms a sensor 7, the number of sensors 7 preferably corresponds to the number of emitters 5.

The sensor device 4 and/or matrix of emitters 5 and detectors 6 preferably has an area of more than 10 cm², in particular more than 20 cm², particularly preferably more than 30 cm², very particularly preferably more than 40 cm², and/or less than 200 cm², preferably less than 150 cm², more preferably less than 100 cm², particularly less than 80 cm².

Preferably, an area density of the emitters 5, an area density of the detectors 6, an area density of the sensors 7 and/or a common area density of the emitters 5 and detectors 6 is more than 0.5/cm², preferably more than 1/cm², in particular more than 2/cm², and/or less than 40/cm², preferably less than 20/cm², in particular less than 10/cm². Herein, the number of emitters 5 and/or detectors 6 and/or sensors 7 per area is in particular denoted as area density.

The number, arrangement, area and/or area density of the sensor device 4, emitters 5, detectors 6 and/or sensors 7 preferably allow a reliable and accurate examination, in particular photoplethysmography and/or determination of blood pressure BP, to be performed without fixation of the paw 2 of the animal T relative to an examination means such as a sensor, so that the animal T can preferably move freely relative to the sensor device 4 during the examination. This makes the examination particularly pleasant and stress-free for the animal T, which improves the measuring accuracy.

The emitters 5 and/or detectors 6 are preferably each divided into several groups or preferably form several groups, which are in particular separately from each other and/or separately connected.

Preferably, the emitters 5 are divided into two groups and/or the emitters 5 form two groups.

Preferably, the detectors 6 are divided into five groups and/or the detectors 6 form five groups.

The emitters 5 within a group and/or the detectors 6 within a group are preferably connected or interconnected serially.

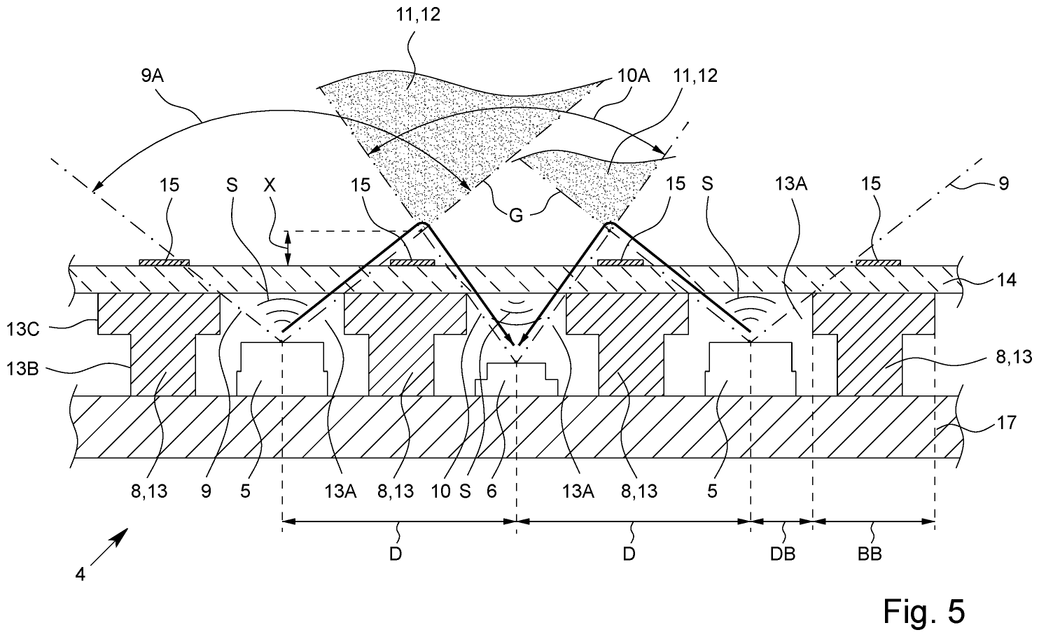

FIG. 5 shows a schematic section through the sensor device 4.

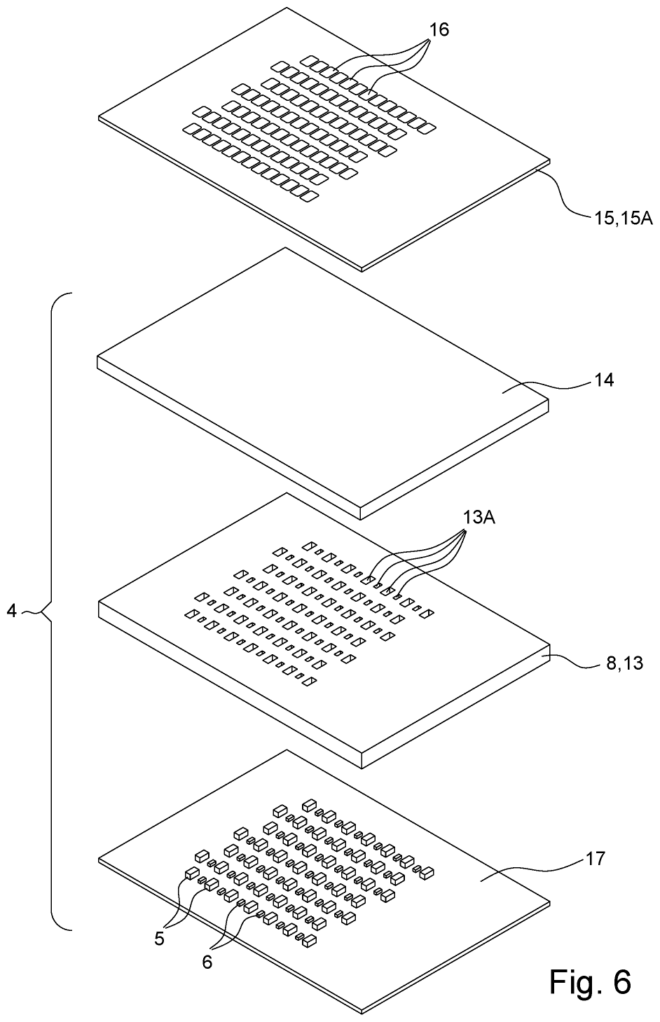

FIG. 6 shows the sensor device 4 in a schematic exploded view.

The sensor device 4 preferably has a limiting device 8.

At this point, it should be noted that the limiting device 8 as well as the associated features and advantages are in principle realizable independently of the above described design of the sensor device 4. In particular, the limiting device 8 can also be advantageous for a sensor device 4 with exactly one emitter 5 and exactly one detector 6. Consequently, the terms “emitter” and “detector” are preferably used in the singular in the following. Of course, the explanations also apply to designs of the sensor device 4 with several emitters 5 and/or several detectors 6, in particular to a sensor device 4 designed as described above.

The limiting device 8 is preferably designed to determine, define and/or limit an emission region 9 of the emitter 5, a detection region 10 of the detector 6, a sensor region 11 of the sensor 7 and/or a sensing region 12 of the sensor device 4. In particular, the limiting device 8 is designed as an aperture for the emitter 5 and/or detector 6.

For this purpose, the limiting device 8 in the illustration example has a barrier 13 described in more detail below or is formed hereby. Alternatively or additionally, however, the limiting device 8 can also have one or more lenses not shown, in particular converging lenses, which lead to a corresponding limitation of an emission region 9 and/or detection region 10, in particular by focusing radiation R.

The emission region 9 of an emitter 5 is generally the range into which radiation R can be emitted by the emitter 5. For example, the emission region 9 of an emitter 5 can be at least essentially conical and/or defined by one or—in particular in the case of a non-conical emission region 9—several emission angle(s) 9A.

The detection region 10 of a detector 6 is generally the range from which radiation R can reach the detector 6 and/or from which radiation R can be detected with the detector 6. For example, the detection region 10 of a detector 6 can be at least essentially conical and/or defined by one or—in particular in the case of a non-conical detection region 10 —several detection angle(s) 10A.

Preferably, the emitter 5 and/or the detector 6 naturally have a certain emission region 9 or detection region 10, respectively. Preferably, this natural emission region 9 and/or detection region 10 is limited or restricted by the limiting device 8 or the limiting device 8 is designed for this purpose. Therefore, the terms “emission region” and “detection region” in the sense of the present invention preferably refer to the emission region 9 or detection region 10 defined or limited by the limiting device 8 and not to the natural emission region 9 or detection region 10 of the emitter 5 or detector 6 per se.

The emission region 9 is indicated in FIG. 5 by the V-shaped dotted lines starting from the emitter 5. The dotted lines represent the border of the emission region 9, which is in particular defined by the limiting device 8. In particular, the emission region 9 is the area enclosed or limited by the lines.

The detection region 10 is indicated in FIG. 5 by the V-shaped dotted lines starting from the detector 6. The dotted lines represent the border of the detection region 10, which is in particular defined the limiting device 8. In particular, the detection region 10 is the area enclosed or limited by the lines.

The emission region 9 of an emitter 5 is preferably limited by (imaginary) lines, in particular those shown in FIG. 5 as dash-dotted lines, which represent the ray path of the outermost rays of a beam of rays that can leave the sensor device 4 starting from a center point or geometric center of an emission region of the emitter 5. In particular, the lines represent an edge or a border of the emission region 9. In particular, the emission region 9 is the region enclosed or limited by the lines.

In case the limiting device 8 is realized by a barrier 13, as shown in FIG. 5, these outermost beams are those beams that are not blocked by the limiting device 8 starting from the center point or geometric center, so that the lines representing these beams in FIG. 5 touch an edge or corner of the limiting device 8 or barrier 13.

If the limiting device 8 has, or is formed by, a lens as an alternative or in addition to the barrier 13, these outermost rays are those rays that pass through the outermost edge of the lens from the center point or geometric center of an emission surface of the emitter 5.

The detection region 10 of a detector 6 is preferably limited by (imaginary) lines, in particular those shown in FIG. 5 as dash-dotted lines, which represent the optical path of the outermost rays of a beam of rays that can reach a detection surface of the detector 6, in particular a center point or geometric center thereof, from outside the sensor device 4. In particular, the lines represent an edge or a border of the detection region 10. In particular, the detection region 10 is the region enclosed or limited by the lines.

In case the limiting device 8 is realized by a barrier 13, as shown in FIG. 5, these outermost rays are those rays that are not blocked by the limiting device 8 and thus can reach the center point or geometric center of the detection surface of the detector 6, so that the lines in FIG. 5 representing these rays touch a rim or edge or corner of the limiting device 8 or barrier 13.

If the limiting device 8 has or is formed by a lens as an alternative or in addition to the barrier 13, these outermost rays are those rays that can pass through the outermost edge of the lens from outside the sensor device 4 and reach the center point or geometric center of the detection surface of the detector 6.

The emission angle 9A is preferably the angle between the (imaginary, in particular outside the sensor device 4 running) lines, which represent the borders of the emission region 9. This is in particular shown in FIG. 5.

Preferably, the detection angle 10A is the angle between the (imaginary, in particular outside the sensor device 4 running) lines that represent the borders of the detection region 10. This is in particular shown in FIG. 5.

In the above definition of the emission region 9 and detection region 10, an idealized approach was chosen, with reference being made to a center point or geometric center of an emission area or detection area, which in reality deviates from a point shape and forms an—albeit very small—extended area. This makes it possible that in reality radiation R from the emitter 5 can also reach an area outside the emission region 9 as defined above and/or radiation R from outside the detection region 10 as defined above can reach the detector 6, in particular scattered light. However, the above definitions of emission region 9 and detection region 10 remain unaffected by this. Furthermore, the emission region 9 and detection region 10 as defined above also represent in reality the region into which the vast majority of the radiation R emitted by the emitter 5 is emitted and/or from which radiation R can reach the detector 6.

The sensor region 11 of a sensor 7 is generally the region that can be examined or sensed with the sensor 7. Preferably, only objects located in sensor region 11 can be examined by means of sensor 7. In particular, the sensor region 11 of a sensor 7 is the region in which the emission region(s) 9 of the emitter(s) 5 of the sensor 7 and the detection region(s) 10 of the detector(s) 6 of the sensor 7 overlap.

In FIG. 5, by way of example, arrows indicate how radiation R can pass from an emitter 5 to a detector 6. The arrows very schematically show the path of a light beam which is emitted by the emitter 5, reaches a detection region 10 and thus a region where the emission region 9 and the detection region 10 overlap, and is scattered or reflected there in the direction of detector 6 by an object not shown and in this way reaches the detector 6.

In principle, it is possible that, deviating from the idealized view chosen here, in reality objects outside the sensor region 11 as defined above are at least partially detected or detectable by a sensor 7. On the one hand, this can take place by the fact that, as already described above, a small amount of radiation R in reality can also reach a region outside the defined emission region 9 and/or radiation R from outside the defined detection region 10 can also reach the detector 6. On the other hand, however, it can also happen, for example in case of multiple scattering in an object, that an object or a part of an object is detected with a sensor 7 which is located outside the defined sensor region 11.

The sensing region 12 of the sensor device 4 is the range which can be examined and/or detected/sensed with the sensor device 4. In particular, the sensing region 12 comprises the emission regions 9, detection regions 10 and/or sensor regions 11 or is formed thereby.

Preferably, the sensing region 12 is the total/entirety of the sensor regions 11 of the sensors 7 of the sensor device 4.

The sensing region 12 can be formed by a continuous/connected region. This is the case if the sensor regions 11 of the sensors 7 of the sensor device 4 overlap.

However, it is also possible that the sensing region 12 is not connected or is formed by separate or non-connected regions or sensor regions 11. This is the case if at least some of the sensor regions 11 of the sensors 7 do not overlap with other sensor regions 11.

The sensing region 12 preferably has a border G. The border G is preferably formed by the edge or the entirety of the edges of the sensor regions 11. The border G is in particular a point or a line where an emission region 9 and a detection region 10 intersect. This is in particular shown in FIG. 5.

The sensing region 12 and/or its border G preferably has a distance X from the sensor device 4. In particular, a (minimum) penetration depth of the radiation R emitted by the emitters 5 and/or detected by the detectors 6 into the paw 2 during the examination can be achieved or ensured. In particular, this minimum penetration depth or distance X prevents light reflected or scattered from a surface of the paw 2 from reaching the detector 6. This improves the accuracy and reliability of the examination, in particular the determination of blood pressure.

The distance X is preferably a minimum distance of the sensing region 12 or its border G from the sensor device 4. Preferably, the border G of the sensing region 12 does not run straight or parallel to the sensor device 4, as can be seen in particular from FIG. 5. In the sectional view as shown in FIG. 5, the border G runs particularly zigzag. This is particularly due to the fact that the sensor regions 11 of the sensors 7 preferably increase (in section) in a V-shape with increasing distance from the sensor device 4. Consequently, the sensing region 12 preferably has different distances from the sensor device 4 at different positions of the sensor device 4, wherein the distance X is the smallest of these different distances.

The limiting device 8 is preferably designed such that the distance X of the border G of the sensing region 12 from the sensor device 4 is more than 0 5 mm, preferably more than 1 mm, and/or less than 10 mm, preferably less than 5 mm, in particular less than 3 mm.

The limiting device 8 preferably limits—in particular in the sectional plane shown in FIG. 5—an emission angle 9A of the emitter 5 and/or a detection angle 10A of the detector 6 to less than 90°, preferably less than 75°, in particular about 60°. The sectional plane shown in FIG. 5 is perpendicular to the plane defined by the matrix of emitters 5 and detectors 6 and intersects the emitters 5 and detectors 6 along a row or column of the matrix.

The limiting device 8 is preferably formed by one or more barriers 13. The barrier 13 is arranged between an emitter 5 and a detector 6. Preferably, a barrier 13 is arranged between each detector 6 and the respective adjacent emitters 5.

The barrier 13 is impermeable to the radiation R emitted by the emitter 5, in particular to infrared radiation.

In principle, however, the limiting device 8 can also be realized differently than by a barrier 13. For example, one or more lenses could be assigned to the emitter(s) 5, which are designed or arranged to focus or scatter the radiation R emitted by the emitter 5 and in this way define the emission region 9 and/or the emission angle 9A. Alternatively or additionally, one or more lenses could be assigned to the detector 6 in a corresponding manner, which are designed or arranged to bundle or scatter the radiation R to be detected by the detector 6, so that the detection region 10 and/or the detection angle 10A is defined.

The barrier 13 is preferably arranged or designed in such a way that the above-mentioned distance X of the border G of the detection range 8 from the sensor device 4 is reached or realized.

The dimensions of the limiting device 8 or barrier 13, in particular its height HB and/or width BB, as well as the distance DB of the limiting device 8 or barrier 13 from the emitter 5 and the detector 6 and the distance D of the emitter 5 from the detector 6 are preferably matched to each other in such a way that the emission region 9 of the emitter 5 and the detection region 10 of the detector 6 overlap in such a way that the above-mentioned distance X of the border G of the sensing region 12 from the sensor device 4 and/or the above-mentioned emission angle 9A and/or detection angle 10A is/are reached or realized.

Preferably, the barrier 13 fulfills several functions and/or has several sections 13B, 13C, which in particular realize these functions.

A function of the barrier 13 is preferably the shielding of the emitter 5 from the detector 6, in particular in such a way that no radiation R emitted by the emitter 5 can reach the detector 6 directly or without intermediate scattering and/or reflection. For this purpose, the barrier 13 preferably has a shielding section 13B. The shielding section 13B is therefore preferably designed to shield the detector 6 from the emitter 5 or to prevent direct crosstalk from the emitter 5 to the detector 6. The shielding section 13B is preferably located between the emitter 5 and the detector 6. The shielding section 13B preferably runs at least substantially parallel to a main emission direction of the emitter 5 and/or transversely, in particular at least substantially perpendicular, to the plane formed by the emitters 5 and detectors 6.

Another function of the barrier 13 is preferably, as already mentioned above, to limit the emission region 9, detection region 10, sensor region 11 and/or sensing region 12. In other words, the barrier 13 and/or a section thereof preferably represents an aperture for the emitter 5 and/or the detector 6. For this purpose, the barrier 13 preferably has an aperture section 13C. The aperture section 13C is preferably designed and/or arranged in such a way that the emission region 9 of the emitter 5 and/or the detection region 10 of the detector 6 is limited or restricted, in particular in the manner described above. The aperture section 13C preferably forms an aperture. In particular, the aperture section 13C preferably runs transversely, preferably at least substantially perpendicularly, to the main emission direction of the emitter 5 and/or at least substantially parallel to the plane formed by the emitters 5 and detectors 6.

The shielding section 13B and the aperture section 13C are preferably designed in one piece and/or formed by different sections of the same component. In particular, the aperture section 13C can be wider than the shielding section 13B, resulting in a T-shaped cross-section of the barrier 13, as shown in FIG. 5. However, this is not mandatory.

The limiting device 8 and/or barrier 13, in particular the aperture section 13C, preferably has a width BB of more than 1 mm, in particular more than 2 mm, and/or less than 5 mm, in particular less than 4 mm. Furthermore, the limiting device 8 and/or barrier 13 preferably has a height HB of more than 1 mm, preferably more than 2 mm, and/or less than 5 mm, in particular less than 4 mm.

Preferably, the barriers 13 form or limit areas 13A that are transparent and/or translucent for the radiation R emitted by the emitters 5 and/or detected by the detectors 6. These transparent areas 13A are each arranged corresponding to the emitters 5 and detectors 6, so that they are located in the sensor device 4 above the emitters 5 and detectors 6, respectively, and the material located between the transparent areas 13A or surrounding the transparent areas 13A forms the limiting device 8 and/or the barriers 13. This is shown as an example in FIGS. 5 and 6.

The examination apparatus 1 and/or sensor device 4 preferably has a barrier element 13D. Preferably, the barrier element 13D has or forms the barrier 13 or barriers 13.

The barrier element 13D is preferably a one-piece, in particular flat and/or plate-like, part having the transparent areas 13A.

The transparent areas 13A are preferably formed by through holes of the barrier element 13D. In principle, however, it is alternatively or additionally possible that the transparent areas 13A are formed by or comprise a material that is transparent for the radiation R emitted by the emitters 5 and/or detected by the detectors 6, for example glass, plexiglass or the like.

In FIG. 6, the transparent areas 13A are shown rectangular. However, deviating from this, the transparent areas 13A can be in particular circular.

The limiting device 8 and/or barriers 13 and/or the barrier element 13D and/or the transparent areas 13A preferably form a grid or grating corresponding to the emitters 5 and/or detectors 6, in particular a grating aperture.

Preferably, the sensor device 4 has a cover 14 which is transparent for the radiation R emitted by the emitter 5 and/or detected by the detector 6. The cover 14 can be made of glass, plexiglass, a transparent plastic or the like.

Preferably, the cover 14 covers the sensor device 4 completely, continuously and/or gaplessly.

The cover 14 is preferably designed to protect the sensor device 4 and/or the emitters 5 and/or detectors 6 from soiling and/or damage. The cover 14 preferably forms or has an at least substantially flat and/or even, in particular smooth, surface to support the paw 2.

Preferably, the cover 14 rests on the limiting device 8 or barrier 13 and/or adjoins thereto, in particular directly. However, it is also possible that the limiting device 8 and/or barrier 13 has or forms the cover 14 and/or that the cover 14 is integrated into the limiting device 8 and/or the barrier 13 and/or the barrier element 13D. In particular, in the case that the transparent areas 13A are formed by or comprise a transparent material, a cover 14 can be formed by the barriers 13 and/or the barrier element 13D at the same time and/or an additional cover 14 can be dispensed with.

Preferably, the sensor device 4 and/or cover 14 is flush with the examination apparatus 1, in particular with the top side of the examination apparatus 1 and/or the rest surface 3, and/or the sensor device 4 and/or cover 14 does not protrude from the rest surface 3 and/or top side.

Particularly preferably, the distance X of the border G of the sensing region 12 from the sensor device 4 is or corresponds to the distance of the border G of the detection zone 12 from the cover 14, in particular the distance from the side of the cover 14 facing away from the emitter 5 and/or detector 6.

The cover 14 is preferably scratch-resistant.

Preferably, the examination apparatus 1 has one or more detection elements for detecting activity of the heart of the animal T, in particular for recording a cardiogram KG.

The cardiogram KG preferably represents an activity of the heart, in particular of the animal T to be examined by means of the examination apparatus 1, and/or comprises information about the activity of the heart.

FIG. 9 shows an example of a cardiogram KG.

In particular, the heartbeats or the times at which the heartbeats can be read or derived or determined from the cardiogram KG.

The cardiogram KG is preferably an electrocardiogram. In principle, however, the cardiogram KG can also be an impedance cardiogram, a phonocardiogram, a ballistocardiogram or the like.

The detection elements are preferably formed by electrodes 15. In principle, however, the detection element(s) can also be formed by or have one or more microphones or other sound sensors or the like.

Preferably, the examination apparatus 1 thus has at least one electrode 15, preferably at least two electrodes 15. In the illustration example, the examination apparatus 1 has three electrodes 15. In principle, however, the examination apparatus 1 can also have a significantly larger number of electrodes 15.

Preferably, a cardiogram KG can be recorded by means of the electrodes 15 and/or the electrodes 15 are designed to record a cardiogram KG, in particular wherein the cardiogram KG is an electrocardiogram.

The electrodes 15 are preferably flat and/or laminar In particular, the electrodes 15 consist of or have an electrically conductive material.

Preferably, at least one of the electrodes 15 is designed as a tissue electrode. This is indicated schematically in FIG. 1 by hatching of electrodes 15. Preferably, all of the electrodes 15 are formed as fabric electrodes. This has proven to be particularly advantageous for the examination of animals T, such as cats or dogs, since the examination can be made particularly pleasant for the animals T as a result. In particular, it has turned out that the animals T are easily irritated by metallic and/or shiny surfaces, which can be avoided by using tissue electrodes.

The at least two electrodes 15 are denoted below as first electrode 15A and second electrode 15B for better differentiation. The electrodes 15A and 15B can be identical or have different designs.

Explanations with reference to the first electrode 15A therefore preferably also apply to the second electrode 15B and vice versa.

Preferably, the electrodes 15A, 15B are each designed to contact a paw 2 of the animal T. Particularly preferably, the first electrode 15A is designed for contacting the left forepaw and the second electrode 15B is designed for contacting the right forepaw.

Optionally, the examination apparatus 1 has a third electrode 15C. The third electrode 15C is preferably designed as reference electrode or collection electrode. The third electrode 15C is preferably designed to simultaneously contact several parts of the body of the animal T to be examined, in particular several paws 2, in particular the two hindpaws of the animal T.

The electrodes 15 are preferably arranged in such a way that when the animal T is placed on the examination apparatus 1, in particular in a position natural for the animal T, such as a sitting or lying position, one paw 2 of the animal T contacts one of the electrodes 15. In this way, the examination can be made particularly pleasant for the animal T.

The arrangement, size and design of the electrodes 15 are preferably adapted to the anatomy of the animal T to be examined, in particular a domestic cat, so that the examination can take place in a natural, preferably pleasant, position for the animal T and/or the animal T can move freely relative to the electrodes 15 during the examination.

The electrodes 15, in particular the first electrode 15A and the second electrode 15B, are preferably arranged at a distance DE of more than 2 cm, in particular more than 5 cm, and/or less than 25 cm, in particular less than 20 cm, particularly preferably less than 15 cm, very particularly preferably about 10 cm.

The distance DE between two electrodes 15 is referred to in particular as the distance DE between the center points or geometric centers of the electrodes 15 or their surface. This is shown schematically in FIG. 1.

The distance DE of the electrodes 15, in particular of the first electrode 15A from the second electrode 15B, is preferably fixed and/or not variable. In other words, the electrodes 15 are preferably arranged at a fixed distance DE from each other and/or cannot be moved relative to each other. Particularly preferably, the distance DE of the electrodes 15, in particular of the first electrode 15A from the second electrode 15B, corresponds to the distance of the forepaws of an animal T, in particular a domestic cat or a domestic dog, in a natural position of the animal T, in particular a sitting and/or lying position, as shown exemplarily in FIG. 2. Hereby, it is possible that the examination of the animal T can be performed in a natural and therefore comfortable position for the animal T. This makes the examination particularly comfortable for the animal T.

The (respective) electrode 15A, 15B preferably has an area of more than 10 cm², in particular more than 15 cm², and/or less than 100 cm², in particular less than 80 cm², particularly preferably less than 50 cm².

The third electrode 15C preferably has an area of more than 50 cm², in particular more than 100 cm², and/or less than 1000 cm², preferably less than 500 cm², in particular less than 200 cm².

The third electrode 15C preferably has a larger area than the first and/or second electrode 15A, 15B, in particular more than double or triple, particularly preferably more than four times, the area of the first and/or second electrode 15A, 15B.

Preferably, the first electrode 15A is arranged in such a way that, at a paw 2, in particular the left or right forepaw, a cardiogram KG can be recorded by means of the first electrode 15A and, simultaneously, the optical examination can be performed and/or the curve K, in particular a photoplethysmogram (PPK), can be recorded by means of the sensor device 4.

FIG. 7 shows by way of example a paw 2 that is positioned in such a way that a cardiogram KG can be recorded by means of the first electrode 15A and, simultaneously, the optical examination can be performed and/or the curve K can be recorded by means of the sensor device 4.

In other words, the first electrode 15A is preferably arranged in such a way that a paw 2 of the animal T can be positioned over the sensor device 4 in such a way that a cardiogram KG can be recorded by means of the first electrode 15A and at the same time the optical examination, in particular photoplethysmography, can be performed on the same paw 2 by means of the sensor device 4.

For this purpose, the first electrode 15A is preferably arranged in the immediate vicinity of the sensor device 4 and/or the emitters 5 and/or the detectors 6 and/or integrated into the sensor device 4. Preferably, the sensor device 4 has the first electrode 15A.

The first electrode 15A is preferably designed as tissue electrode.

A tissue electrode is preferably an electrode that has or is formed by a tissue. In particular, in the case of a tissue electrode, a contact surface for contact with a body part, in particular the paw 2, has a tissue or is formed hereby. The tissue is preferably a conductive tissue, for example a tissue in which conductive threads are incorporated and/or a tissue coated with a conductive layer.

The first electrode 15A is preferably arranged on the sensor device 4 and/or on the cover 14, particularly preferably on the side of the cover 14 facing away from the emitter 5 and detector 6. This is in particular shown in FIGS. 5 to 7.

However, if no cover 14 is provided, the first electrode 15A may also be arranged directly on the limiting device 8 and/or barrier 13 and/or have or form the cover 14 or a part thereof.

The first electrode 15A is preferably arranged (only) between the emitter 5 and the detector 6 and/or opposite the barrier 13 in a projection perpendicular to the cover 14 and/or to the plane formed by the emitters 5 and detectors 6. Alternatively, or additionally, the electrode 15A is transparent for the radiation R emitted by the emitter 5. Hereby, the optical examination of the animal T and/or the paw 2 by means of the sensor device 4 is not affected by the first electrode 15A.

Preferably, the first electrode 15A is formed in one piece, especially flat, plate-like or plate-shaped and/or mat-like or mat-shaped.

The first electrode 15A preferably has areas 16 that are transparent to the radiation R emitted by the emitters 5 and/or detected by the detectors 6. These transparent areas 16 are arranged corresponding to the emitters 5 and detectors 6, so that they are located (in a projection perpendicular to the plane of the emitters 5 and/or detectors 6 and/or to the cover 14) above the emitters 5 and detectors 6, respectively.

This is in particular shown in FIGS. 5 and 6.

The transparent areas 13A of the first electrode 15A are preferably formed by through holes of the electrode 15A. In principle, it is alternatively or additionally possible that the transparent areas 16 or the entire first electrode 15A are formed by or comprise a material that is transparent for the radiation R emitted by the emitters 5 and/or detected by the detectors 6.

The first electrode 15A and/or the transparent areas 16 preferably form a grating or grid corresponding to the emitters 5 and/or detectors 6.

In particular, as an alternative or in addition to the limiting device 8 and/or barrier 13, the electrode 15A can be designed, in particular by means of the transparent areas 13A and the intransparent material arranged in between, to limit or define the emission regions 9 and/or detection regions 10. In particular, the first electrode 15A may form or have one or more apertures for the emitters 5 and/or detectors 6. In this sense, the electrode 15A may in particular form or have the limiting device 8 and/or barrier 13 or a part thereof.

The electrodes 15 are preferably designed to be scratch-resistant, especially in such a way that they cannot be scratched by a domestic cat or dog to be examined or their claws.

The electrodes 15 may be produced and/or applied to the examination apparatus 1 and/or the sensor device 4, in particular the cover 14 or barrier 13, by gluing, printing, spraying, vapor deposition (in particular physical vapor deposition (PVD)), chemical vapor deposition, in particular plasma-assisted chemical vapor deposition, selective electroplating, indium tin oxide coating, doping a transparent carrier material with electrically conductive particles or the like.

Optionally, the examination apparatus 1 has a positioning aid 24. The positioning aid 24 is designed to support correct positioning of the animal T or the paw 2 for examination. In particular, the positioning aid 24 is designed to indicate or mark an area for positioning a paw 2 or several paws 2, in particular the left forepaw and/or the right forepaw. The positioning aid 24 is preferably arranged near the sensor device 4 and/or preferably surrounds the sensor device 4. Alternatively or additionally, the position of one or more of the electrodes 15 can be indicated by the positioning aid 24.

The positioning aid 24 is preferably formed by an elevation or recess of the examination apparatus 1 and/or rest surface 3. The positioning aid 24 can, for example, be funnel-like or have the shape of a funnel.

However, the positioning aid 24 is only optional and not mandatory.

Optionally, the examination apparatus 1 can also have a feeding place not shown in the figures, by which the animal T is fed or can be fed during the examination. For example, the feeding place may have or be formed by a bowl or cup for food and/or a drinking bottle.

The examination apparatus 1 preferably has a circuit board 17, in particular a printed circuit board (PCB).

Preferably, the circuit board 17 carries the sensor device 4 and/or the sensor device 4 is located on the circuit board 17.

Preferably, the circuit board 17 carries the first and/or second electrode 15A, 15B or the first and/or second electrode 15A, 15B are arranged on the circuit board 17. Optionally, the circuit board 17 carries additionally also the third electrode 15C and/or the third electrode 15C is also arranged on the circuit board 17.

The circuit board 17 preferably has or forms peripherals and/or electrical lines required for the operation of the sensor device 4, in particular the emitters 5 and/or detectors 6 and/or sensors 7, and/or the electrodes 15A, 15B and/or for the evaluation of the signals measured by the detectors 6 and/or electrodes 15.

The examination apparatus 1 preferably has a scale 18. The scale 18 is preferably an electronic scale 18.

The scale 18 is preferably designed for weighing an animal T positioned or placed on the examination apparatus 1.

The examination apparatus 1 and/or scale 18 is preferably designed for a body fat measurement, i.e. for determining the body fat percentage of the animal T on the scale 18. The body fat measurement or determination of the body fat percentage is preferably carried out via a bioimpedance measurement. In particular, two or more of the electrodes 15, 15A, 15B, 15C can be used for this purpose.

The examination apparatus 1 preferably has a force sensor 18A. The force sensor 18A is preferably designed to measure or detect a force, in particular a weight force, exerted by the animal T on the examination apparatus 1.

The force sensor 18A can form part of the scale 18 or be integrated into the scale 18, but can also be provided as an alternative or in addition to the scale 18.

The force sensor 18A can, for example, be designed as a piezo element or strain gauge or the like.

The examination apparatus 1 can also have several force sensors 18A, in particular of the same kind or type. Preferably, one or more force sensors 18A are arranged under the sensor device 4 or the sensor devices 4, under the rest surface 3 and/or under the electrodes 15 (each) and/or the force sensors 18A are integrated into the sensor device(s) 4 and/or rest surface 3 and/or electrodes 15. In particular, the force sensor 18A can be designed by such an arrangement to determine a presence and/or positioning of the animal T and/or to support such a determination.

The examination apparatus 1 preferably has a display device 19. The display device 19 is in particular designed for optical display. The display device 19 is preferably formed by a display, e.g. an LCD display, an LED display, an OLED display or the like.

The display device 19 is preferably designed to display values measured or determined by means of the examination apparatus 1, such as a cardiogram KG, a heart rate, a blood pressure BP, a weight, a body fat percentage or the like. In particular, the display of a blood pressure BP and a cardiogram KG by means of the display device 19 are shown schematically in FIG. 1.

Alternatively, or additionally, the display device 19 can be designed for user guidance, e.g. to display instructions for the operation or use of the examination apparatus 1, selection menus, error messages, warning messages or the like.

Furthermore, the examination apparatus 1 preferably has an input device 20. The input device 20 is preferably designed for making settings and/or adjustments and/or for controlling the examination apparatus 1. The input device 20 is preferably arranged in the immediate vicinity of the display device 19 and/or integrated into the display device 19.

For example, the input device 20 can be formed by one or more keys, buttons, switches, or the like. However, the display device 19 is particularly preferably designed as a touch display or touch-sensitive display, so that the display device 19 has or forms the input device 20 and/or the input device 20 is integrated into the display device 19.

The examination apparatus 1 preferably has a power supply device 21. The power supply device 21 is designed to supply the examination apparatus 1 with electrical energy.

Preferably, the power supply device 21 has an energy storage device for storing electrical energy, for example an accumulator, a battery or the like. In particular, the power supply device 21 is designed for charging the accumulator or battery, particularly preferably for inductive charging. For this purpose, the power supply device 21 preferably has a corresponding charging device. Alternatively or additionally, the power supply device 21 can also have or form a connection for connecting the power supply device 21 to an external power supply, e.g. the mains. In particular, the connection can comprise or form the charging device or a part thereof.

The examination apparatus 1 preferably has a control device 25 for controlling the examination apparatus 1 and/or the examination. The control device 25 is preferably formed by a processor P and/or preferably has a processor P. The processor P is preferably a microprocessor. The control device 25 and/or the processor P is/are preferably designed to control the sensor device 4, in particular the emitters 5, detectors 6 and/or sensors 7, to control the electrodes 15 and/or to control the scale 18.

Accordingly, the control device 25 is preferably coupled with the sensor device 4, the emitters 5, the detectors 6, the sensors 7, the electrodes 15, the scale 18 and/or the force sensor 18A.

Furthermore, the power supply device 21 is preferably designed to supply power to the control device 25. In particular, the control device 25 is coupled to the power supply device 21.

The control device 25 is preferably designed to control the display device 19 and/or coupled to the display device 19. Preferably, the control device 25 is coupled to the input device 20 and/or can be operated by means of the input device 20.

The control device 25 is preferably designed for processing and/or forwarding the signals measured by the sensor device 4 and/or the electrodes 15.

The examination apparatus 1 preferably has a memory and/or a storage medium 26 for data storage. Preferably, the storage medium 26 is coupled with the control device 25. In particular, the storage medium 26 is designed for at least temporary storage of signals measured by the sensor device 4 and/or the electrodes 15.

The storage medium 26 can have several separate components and/or be formed hereby.

Preferably, the storage medium 26 has one or more permanently installed memory modules and/or storage elements, for example a hard disk (HDD), a solid-state drive (SSD), a RAM module and/or a flash memory or the like.

Alternatively, or additionally, the storage medium 26 may have or be formed by one or more storage elements that are separate from and/or connectable to the examination apparatus 1, such as a USB stick or the like.

In principle, the storage medium 26 may be formed by or comprise one or more arbitrary storage devices for storing electronic data, such as CD-ROMs, hard disks, USB sticks, flash memory, cloud memory, external databases or other computer equipment separate from the examination apparatus 1 or external thereto and/or mobile end devices with an integrated memory, such as PCs, data centers, supercomputers, cloud computers, servers, cell phones, smart phones, tablets, laptops or the like.

The examination apparatus 1 is preferably designed for the analysis and/or evaluation of the signals measured with the electrodes 15, the sensor device 4 and/or the scale 18. The evaluation of the signals is preferably performed by means of the control device 25 and/or the processor P and/or is controlled hereby, in particular by using the storage medium 26.

The examination apparatus 1 preferably has an interface device 22 for connecting the examination apparatus 1 with one or more external devices 23. The interface device 22 may have several, in particular different, interfaces. The interfaces can be wired or wireless interfaces. For example, the interface device can have one or more serial interfaces, one or more USB interfaces, one or more HDMI interfaces and/or some or more other interfaces, which are in particular designed for (in particular wired) data exchange between the external device 23 and the examination apparatus 1. Alternatively, or additionally, the interface device 22 may also have one or more wireless interfaces, such as Wi-Fi interfaces, Bluetooth interfaces, in particular Bluetooth Low Energy Interfaces (BLE interfaces), NFC interfaces or the like.

In other words, the examination apparatus 1 is preferably designed for data exchange with an external device 23, in particular by means of the interface device 22.

The examination apparatus 1 is preferably designed to transmit the data or signals measured with the sensor device 4 and/or the electrodes 15 and/or the results or evaluations determined on the basis of these data or signals to the external device 23, in particular by means of the interface device 22.

The external device 23 is preferably a device that is separate, in particular physically separate, from the examination apparatus 1.

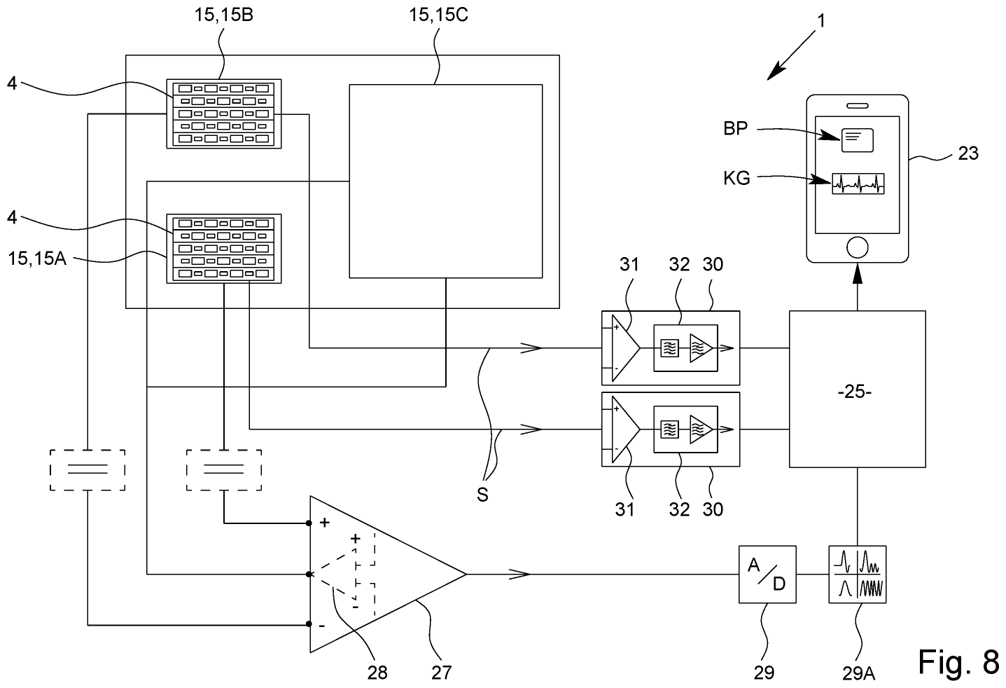

The external device 23 may be designed to control the examination apparatus 1 and/or to record and/or evaluate and/or analyze and/or display or otherwise output signals and/or data measured by the examination apparatus 1 and/or results transmitted by the examination apparatus 1. Preferably, the external device 23 is designed to display a cardiogram KG and/or a blood pressure BP, as shown schematically in FIG. 8.

The external device 23 is preferably designed as a mobile end device, for example a smartphone, tablet or laptop, and/or as a PC, server, computer network, cloud, Internet portal, app and/or other computer device.

Alternatively, or additionally, the external device 23 is designed as a storage medium 26 such as a memory stick. In particular, the external device 23 can form or have the storage medium 26 or a part thereof.

Preferably, the examination apparatus 1 has the external device 23 or the external device 23 forms a part of the examination apparatus 1 or the external device 23 is assigned to the examination apparatus 1.

Preferably, an evaluation of the signals measured by the examination apparatus 1, in particular by the sensor device 4 and/or the electrodes 15, 15A, 15B, 15C, is performed in or by the examination apparatus 1 itself. Alternatively, or additionally, the evaluation or parts thereof can also take place outside the examination apparatus 1 and/or by means of the external device 23.

In FIG. 8, a wiring of the electrodes 15 as well as a processing of the signals measured by the sensor device(s) 4 and the electrodes 15 are shown in a schematic, block diagram-like representation.

The examination apparatus 1 preferably has a preprocessing device 27. The preprocessing device 27 preferably has or is formed by an amplifier, in particular a differential amplifier. The differential amplifier is particularly preferably formed by an operational amplifier or has such an amplifier. However, other solutions are also possible.

The preprocessing device 27 is preferably coupled or connected to the electrodes 15 and is in particular designed for preprocessing the signals measured by the electrodes 15, 15A, 15B, 15C. In particular, the preprocessing device 27 is designed to amplify the difference between signals measured with different electrodes 15, in particular voltages such as biopotentials, particularly preferably to amplify the difference between the signal measured with the first electrode 15A and the signal measured with the second electrode 15B.

Optionally, the electrodes 15 are coupled to the preprocessing device 27 via a capacitance or a capacitor. This is indicated in FIG. 8 by the capacitance symbols in dotted boxes.

Furthermore, the preprocessing device 27 is preferably designed for filtering the signals measured by the electrodes 15.

Preferably, but only optionally, the preprocessing device 27 has a common mode suppression device 28.

The common mode suppression device 28 is preferably designed to suppress or filter out a DC current component or DC voltage component of the signals measured by the various electrodes 15.

The examination apparatus 1 preferably has an A/D converter 29. The A/D converter 29 is preferably designed to convert, in particular, an analog signal, preprocessed by the electrodes 15 and possibly by the preprocessing device 27, into a digital signal. The A/D converter 29 is preferably downstream of the preprocessing device 27.

The signal measured with the electrodes 15, in particular the cardiogram KG recorded with electrodes 15, is preferably further evaluated and/or processed, in particular after conversion into a digital signal. In particular, a usefulness check can be performed, e.g., by a check device 29A. During the usefulness check, it is preferably determined whether the cardiogram KG is useful, i.e. whether it can be meaningfully evaluated and/or contains useful information. This is shown schematically in FIG. 8 by the box in the lower right corner.

Preferably, the examination apparatus 1, as an alternative or in addition to the preprocessing device 27, has one or more further preprocessing devices 30. The preprocessing device 30 is preferably designed for the preprocessing of signals S measured by the sensor device 4 or detectors 6 and/or sensors 7.

The preprocessing device 30 preferably has an amplifier 31. The amplifier 31 is preferably designed to amplify a signal S measured by a detector 6 or sensor 7. In particular, the amplifier 31 is a transimpedance amplifier and/or converts a current into a voltage.

Preferably, the preprocessing device 30 has a filter device 32 for filtering the signal S, which is in particular amplified by the amplifier 31.

The filter device 32 preferably has several different electrical filters. In particular, the filter device 32 may have or form one or more passive filters and/or one or more active filters. The filter device 32 may, for example, comprise or form one or more bandpass filters, bandstop filters, high-pass filters and/or low-pass filters.

Preferably, each detector 6 or sensor 7 is assigned a preprocessing device 30 or each detector 6 or sensor 7 has a preprocessing device 30.

Preferably, an evaluation of the signals S measured by the sensor device 4 and preferably preprocessed by the preprocessing device 30, in particular the curves K, is performed together with the cardiogram KG and/or under consideration of the cardiogram KG.

The result of the evaluation can then, for example, be forwarded to an external device 23, as already described above and schematically indicated in FIG. 8.

The examination apparatus 1 is preferably designed to perform the method described below. Alternatively, or additionally, the examination apparatus 1 can be used to perform the method described below. This use can also be realized independently of further aspects of the present invention.

In particular, the examination apparatus 1 has means to perform the steps of the method. These means preferably comprise or are formed by a computer program.

According to another aspect, the computer program and/or the instructions are stored on computer-readable storage medium 26 or the computer-readable storage medium 26 comprises the computer program and/or instructions.

The means and/or computer program preferably comprise instructions which, when executed, cause the test apparatus 1 to perform the described method.

For medical examination, in particular blood pressure determination, by means of the examination apparatus 1, it is preferably intended that the animal T, in particular a domestic cat or a domestic dog, is placed on the examination apparatus 1. In particular, the animal T is placed completely on the examination apparatus 1, i.e., preferably in such a way that all limbs, in particular paws 2, are on the examination apparatus 1 and/or the entire weight of the animal T is carried by the examination apparatus 1.

Particularly preferably, the animal T is positioned on the examination apparatus 1 in such a way that a paw 2, in particular a forepaw, of the animal T rests on the sensor device 4 and/or is positioned directly above the sensor device 4 and/or a curve K comprising information about the arterial blood flow BF can be recorded on the paw 2.

Preferably, the animal T is positioned in such a way that each of the electrodes 15, 15A, 15B, 15C contacts a body part, in particular a paw 2, of the animal T, so that a cardiogram KG can be recorded by means of the electrodes 15. In particular, the animal T is positioned so that one of the forepaws contacts the first electrode 15A, the other forepaw contacts the second electrode 15B and, if the examination apparatus 1 has a third electrode 15C, one or both hindpaws contact the third electrode 15C.

After positioning the animal T, the medical examination and/or blood pressure determination is preferably started. Optionally it can be provided that after the positioning of the animal T first of all it is shortly awaited, so that the animal T can calm down and only after a waiting period the medical examination and/or blood pressure determination is begun. In particular, a curve K is recorded for the medical examination or blood pressure determination, which comprises information about an arterial blood flow BF of the animal T. This curve K is in particular a photoplethysmogram.

In the bottom of FIG. 9, a curve K is shown as an example.

Particularly preferably, a reflection measurement is performed for recording the curve K, or the examination apparatus 1 is designed for this purpose. This means in particular that the sensor device 4 is only located on one side of the paw 2 and/or has no components located on opposite sides of the paw 2.

Preferably, the examination or measurement is performed with radiation R in the infrared range.

It is particularly preferable that a cardiogram KG of animal T is recorded by means of the examination apparatus 1, in particular at the same time as the recording of the curve K comprising information about the arterial blood flow BF of the animal T.

In the top of FIG. 9, a cardiogram KG is shown as an example.

Preferably, a presence and/or positioning of the animal T can be or is determined by means of the examination apparatus 1. In particular, this is done by evaluating signals measured with the sensor device 4, the electrodes 15 and/or the scale 18. Preferably, the determination of the presence and/or positioning of the animal T is done before recording the curve K comprising information about the arterial blood flow BF. However, the determination of presence and/or positioning is not mandatory and can also be omitted.

The determination of the presence and/or positioning of the animal T is preferably done in several steps.

In a first step, it is preferably determined whether there is an animal T on the examination apparatus 1 at all. Optionally, the examination apparatus 1 can automatically switch from an energy-saving mode to an operating mode when the presence is detected.

In a second step, which may also be carried out simultaneously with the first step, it is preferably checked or determined whether the animal T is positioned on the examination apparatus 1 in such a way that the medical examination can be performed.

In a third step, which can also be carried out simultaneously with the first and/or second step or instead of the second step, it is preferably determined over which of the sensors 7 of the sensor device 4 a paw 2 or another body part of the animal T is positioned and/or with which of the sensors 7 of the sensor device 4 the medical examination can be carried out.

Preferably, the presence and/or positioning of the animal T is determined by means of the electrodes 15. This is done in particular by a resistance measurement. The resistance measured with the electrodes 15 changes in particular depending on whether or not the electrodes 15 are contacted by a paw 2 of the animal T. In this way, it can be determined whether and/or to which of the electrodes 15 a paw 2 of the animal T is in contact. Hereby, it can be determined whether the animal T is correctly and/or completely positioned on the examination apparatus 1, in particular in such a way that a cardiogram KG can be recorded by means of the electrodes 15.

Alternatively, or additionally, the presence of the animal T can be determined by means of the scale 18 and/or the force sensor 18A. In particular, a force or weight threshold value can be specified or specifiable for this purpose. In this case, the force or weight threshold value is preferably selected in such a way that it is exceeded when a domestic cat or a domestic dog or any other animal T to be examined is placed on the examination apparatus 1. Therefore, exceeding a weight threshold value is an indication of the presence of the animal T. Falling below the weight threshold value is an indication that no animal T is positioned on the examination apparatus 1 and/or that the animal T is only partially positioned on the examination apparatus 1 or not positioned on the examination apparatus 1 in the intended manner.

By means of an appropriate arrangement of the force sensor(s) 18A it is preferably also possible to determine by means of the force sensor(s) 18A whether and/or which of the electrodes 15 and/or sensor device(s) 4 are contacted by the animal T.

Alternatively, or additionally, it can be determined by means of the sensor device 4 whether a paw 2 or any other part of the body of the animal T is located directly above the sensor device 4 and/or whether it is arranged in such a way that the paw 2 and/or the body part can be examined optically by means of the sensor device 4, in particular whether a photoplethysmography can be performed. This is preferably done by comparing the signals S measured by the sensors 7 of sensor device 4.

The comparison of signals S measured with the sensors 7 and/or detectors 6 is preferably done with activated or switched-on or emitting emitters 5, but can also be done with switched-off emitters 5.

By comparing the signals S from different sensors 7 and/or detectors 6 it can preferably be determined in which position the paw 2 is located. In particular, the shape and/or positioning of the paw 2 can preferably be modelled.

If a paw 2 is located on the sensor device 4, preferably some areas of the sensor device 4 and/or some sensors 7 are covered by the paw 2 and other areas and/or sensors 7 are not covered by the paw 2. In particular, this leads to differences in the brightness and/or radiation R measured by the individual sensors 7. For the examination by means of the sensor device 4, it is preferably intended that a paw 2 is positioned over the sensor device 4 in such a way that the sensor 7 or at least one sensor 7 is completely covered by the paw 2. In this way, no ambient light can reach the sensor 7 or its detector 6, but only radiation R that was emitted by the emitter 5 or one of the emitters 5 of the sensor 7 and scattered in the paw 2 towards the detector 6.

The comparison of the different sensors 7 and/or the signals S measured with the sensors 7 is preferably done by forming differences between the signals S of different sensors 7.

Alternatively, or additionally, a position or presence determination by means of the sensor device 4 can be carried out by examining a signal S measured by means of the sensor device 4 to see whether it exceeds or falls below a threshold value, in particular an absolute signal strength.

Preferably, the threshold value represents an absolute brightness. In this way, it can in particular be determined whether a paw 2 and/or any other body part of the animal T is located above a sensor 7 of the sensor device 4 and/or above which sensors 7 of the sensor device 4 a paw 2 or any other body part is located.

In particular, exceeding the threshold value is an indication that no part of the body of the animal T is above the sensor device 4 or the sensor 7 and/or falling below the threshold value is an indication that the paw 2 or another part of the body of the animal T is located above the sensor device 4 and/or the sensor 7 in such a way that the curve K can be recorded.

Alternatively, or additionally, it can be provided that the wavelength of the radiation R measured by detector 6 or sensor 7 is analyzed. Preferably, the emitters 5 are designed to emit radiation R of a certain wavelength or in a narrow wavelength range. In other words, the emitters 5 preferably have a narrow spectrum. In contrast, ambient light, such as sunlight and/or artificially generated light for indoor lighting, usually has a wide spectrum, i.e. a plurality of different wavelengths, which are particularly outside the wavelength range emitted by the emitter 5. Therefore, by spectral analysis of the radiation R detected by the detector 6 or sensor 7, it can preferably be determined whether the sensor 7 is covered by a paw 2 or ambient light is measured.

If it is found that the paw 2 is located only above some sensors 7 of the sensor device 4, in particular thus not over all sensors 7 of the sensor device 4, these sensors 7 can be selected for performing the examination and/or for recording a curve K comprising information about the arterial blood flow BF.

For presence and/or position determination by means of the sensor device 4, in particular a scan or search run can be performed by means of the sensors 7, in which different sensors 7 and/or emitters 5 are activated or switched on one after the other. In particular, the influence of ambient light can be determined hereby and/or by comparing a signal S measured with the emitter 5 switched on with a signal S measured with the emitter 5 switched off.

After the presence and/or position determination and/or after the sensor selection, preferably the medical examination, in particular blood pressure determination, follows by means of the sensor device 4 and/or the electrodes 15, thus particularly preferably the recording of a curve K comprising information about the arterial blood flow BF by means of the sensor device 4 and/or the recording of a cardiogram KG by means of the electrodes 15. The medical examination preferably follows only if the presence and/or position determination has shown that an animal T is positioned on the examination apparatus 1 in such a way that the medical examination can be carried out by means of the sensor device 4 and/or electrodes 15. Preferably, the examination is started automatically if the presence and/or position detection is successful.

However, the examination can also be performed without presence and/or position detection and/or sensor selection.

In particular, a curve K comprising information about an arterial blood flow BF of animal T is recorded by means of the sensor device 4. This is done by positioning the paw 2 over the sensor device 4 in such a way that the radiation R emitted by one or more emitters 5 enters the paw 2 and is scattered and/or reflected to one or more detectors 6. In particular, the time course of the signal S picked up by the detector 6 and/or sensor 7 is recorded.

Preferably, the time course of the signal S recorded by a detector 6 and/or sensor 7 is referred to as the curve K, in particular as a photoplethysmogram (PPG).

The radiation R emitted by the emitters 5 is scattered and/or reflected within the paw 2 during the examination of the paw 2 and can thus reach a detector 6. This is shown as an example in FIG. 7. The signal S measured by the detector 6 thus corresponds to the scattering, reflection and/or absorption of the radiation R emitted by the emitters 5 within the paw 2. Here, the scattering, reflection and/or absorption depends among other things on the volume of the blood in the blood vessels running in the paw 2 and/or on the oxygen saturation of the blood.

The scattering, reflection and/or absorption and thus the curve K measured by the detector 6 and/or sensor 7 are composed of a temporally at least approximately constant component and a temporally varying component.

The temporally constant the time course of the signal S recorded by a detector 6 or sensor 7 is caused in particular by the tissue surrounding the blood vessels, such as muscles, nerves, tendons, bones and/or skin, as the scattering and/or absorption by this tissue preferably does not change or only changes to a small extent. In particular, this temporally at least approximately constant component is not correlated with the heartbeat of animal T. The blood flowing through the veins can also contribute to this at least approximately constant component.

The temporally varying component is preferably caused, at least essentially, by the temporal change of the arterial blood flow BF, i.e., the blood flowing through arteries A. Arteries A are blood vessels through which the blood is carried away from the heart. The blood volume or volume flow through the arteries A and the oxygen saturation of the blood in arteries A change in a way correlated with the heartbeats. In particular, the absorption and/or scattering of blood in the arteries A does not only depend on the blood volume or blood flow in the arteries A, but also on the oxygen content or oxygen saturation of the blood in the arteries A.

Preferably, a curve feature is determined by means of the curve K. The curve feature is in particular a pulse transit time, particularly preferably the time interval between a heartbeat and the arrival of the pulse wave caused by this heartbeat at a specific location of an artery A. Here, the pressure wave that passes through the arteries A is referred to as the pulse wave.

In principle, however, another curve feature can be used instead of the pulse transit time. The curve feature is preferably a feature of curve K or a curve section KA that is related to a pulse transit time and/or a blood pressure and/or correlated with a pulse transit time and/or a blood pressure. In particular, a curve feature is a feature by means of which the blood pressure can be determined. The curve feature is particularly preferably a feature of the curve K and/or the curve section KA that corresponds to a course of the curve K and/or the curve section KA and/or contains information about a shape of the curve K and/or the curve section KA.

For the determination of the curve feature and/or the pulse transit time, it is advantageous to record a cardiogram KG simultaneously with the curve K. This facilitates in particular the determination of the heartbeat and/or the time at which the pulse wave starts at the heart. In principle, however, it is also possible to determine the curve feature or the pulse transit time without recording a cardiogram KG at the same time, for example, by an autocorrelation of the curve K or the like.

The curve K is preferably cut into curve sections KA. This is in particular done in such a way that the curve sections KA correspond to heartbeats, preferably in such a way that each curve section KA corresponds to exactly one heartbeat. Here, however, other solutions are also possible. Particularly preferably, a curve section KA starts at the time of a first heartbeat and ends at the time of a further heartbeat immediately following the first heartbeat.

The cutting of the curve K into curve sections KA is preferably automated or takes place in an automated manner.

Particularly preferably, the curve K is cut into the curve sections KA using information from the cardiogram KG recorded at the same time as the curve K. In principle, however, other methods are also conceivable here.

The use of the cardiogram KG to slice/cut the curve K into curve sections KA is particularly advantageous because the times TH of heartbeats can be determined particularly easily and reliably in a cardiogram KG and the curve K can be cut at or based on these times TH.

Preferably, the times TH of heartbeats are determined on the basis of the cardiogram KG and the curve K at these times TH is cut into curve sections KA. Preferably, each curve section KA starts at the time TH of one heartbeat and ends at the time TH of the immediately following next heartbeat.

In FIG. 9, different QRS complexes of a cardiogram KG are marked. One QRS complex preferably represents one heartbeat.

Preferably, the positions of one or more of the QRS complexes of the cardiogram KG are used to cut the curve K into curve sections KA. In particular, the QRS complexes of the cardiogram KG are used to determine the time TH of heartbeats, preferably wherein the curve K is cut into curve sections KA at the times TH determined by means of the QRS complexes. In other words, the QRS complexes or parts thereof are information by means of which cut the curve K is cut into sections KA.

A QRS complex preferably has three peaks, in particular a Q peak, an R peak and an S peak.

As Q peak is denoted the first, in particular negative or downward pointing, deflection or peak of the QRS complex.

As R peak is denoted the, in particular negative or downward pointing, deflection or peak of the QRS complex which follows the Q peak.

As S peak is denoted, in particular, the positive or upward pointing, deflection or peak of the QRS complex which follows the R peak.

In particular, the position of the R peak or of the maximum of the R peak can be used as time TH of the heartbeat. This is shown by way of example in FIG. 9.

As an alternative to using the R peak as the time TH of the heartbeat, it is also conceivable to use another structure or another characteristic point of the cardiogram KG as the time TH of the heartbeat, for example the Q peak, the S peak, a midpoint or inflection point between two peaks, in particular the R peak and the S peak, or the like.

Preferably, the curve feature and/or the pulse transit time is determined by means of the curve K. This is done in particular on the basis of a plurality or large number of curve sections KA.

Alternatively, or additionally to the determination of the pulse transit time, the pulse wave velocity can be determined. The pulse wave velocity is the quotient of the distance travelled by the pulse wave and the pulse transit time required to travel this distance. In particular, the pulse wave velocity can be used instead of the pulse transit time as a variable in a correlation function to determine the blood pressure BP from the pulse transit time and/or can be considered in the correlation function in addition to the pulse transit time.

Preferably, an averaging based on several curve sections KA is performed for the determination of the curve feature and/or the pulse transit time.

An “averaging” in this sense is in particular the determination of a mean or average course of a set of several curve sections KA or a mean or average course of the curve K during a heartbeat.

In the averaging, in particular a curve mean value is determined. The curve mean value is in particular the mean or average course of a curve section KA or the curve K in a curve section KA. In particular, the curve mean value is determined by calculating for the respective point of time the mean value of the curve sections KA at this point of time. This mean value is preferably the arithmetic mean, but can also be another mean value.

From the curve feature and/or the pulse transit time or on the basis of thereof, the blood pressure BP of animal T is preferably determined, in particular by means of a correlation function. The correlation function can be determined empirically, for example.

The correlation function therefore preferably represents a link between the curve feature or the pulse transit time and the blood pressure BP and/or assigns a blood pressure BP to the curve feature or pulse transit time.

In the context of the present invention, it has been shown that the pulse transit time in animals T, in particular domestic cats and domestic dogs, is correlated with the blood pressure BP.

The correlation function is preferably a scalar field dependent on at least two variables.

Preferably, the curve feature or the pulse transit time constitutes a variable of the correlation function.

It is preferred that in addition to the curve feature or the pulse transit time, a heart rate constitutes a variable of the correlation function. The heart rate describes the number of heartbeats in a certain time interval and is preferably determined from the cardiogram KG, in particular from the distance of QRS complexes or R peaks.

The correlation function can thus, for example, take the functional form:

F(x,y)=a·x+b·y+c

wherein x represents the pulse transit time, y represents the heart rate and a, b, and c are parameters to be determined.

Furthermore, the correlation function is preferably a nonlinear function. The correlation function can thus depend in a nonlinear way on the pulse transit time and/or the heart rate, in particular it can thus have higher order terms in x and/or y (such as x², x³, y², y³, etc.)

In principle, the correlation function can also depend on anatomical peculiarities of the respective animal T For example, it may be provided that a leg or arm length or any other parameter corresponding to a distance between the heart and the paw 2, is taken into account in the correlation function. A preferred parameter in this context may also be the weight of the animal T, since in many cases this allows to draw sufficiently accurate conclusions about the distance between heart and paw 2. In this respect, the correlation function can thus have the weight of the animal T as a parameter.

Complementarily, a parameter corresponding to the body fat percentage, such as the bioimpedance, can be taken into account. A respective measurement can be made using the electrodes 15 for determining the cardiogram KG and/or the scale 18. In particular, the combination of the bioimpedance with the weight of the animal T can, taken into account in the correlation function F by implicit or actual conclusions about anatomical peculiarities of the animal T with regard to the distance between heart and paw 2, make possible a more reliable determination of the blood pressure BP from the pulse transit time.

Further aspects of the present invention which are realizable independently or in combination with the aspects and features described above are in particular:

1. Examination apparatus 1 for medical examination, in particular determination of a blood pressure BP, of an animal T, in particular an animal T having a paw 2, particularly preferably an animal T from the subfamily of the Felinae, very particularly preferably a domestic cat,
with a sensor device 4 for optical examination of an arterial blood flow BF of the animal T, in particular for performing a photoplethysmography, wherein the sensor device 4 has at least one emitter 5 for emitting electromagnetic radiation R and at least one detector 6 for detecting the radiation R emitted by the emitter 5,
characterized
in that the sensor device 4 has a several emitters 5 and several detectors 6, the emitters 5 and detectors 6 being arranged in a periodic structure, and/or in that the sensor device 4 has a limiting device 8 which defines a border G of a sensing region 12 of the sensor device 4, so that a distance X of the border G from the sensor device 4 is more than 0.5 mm and/or less than 5 mm.
2. Examination apparatus according to aspect 1, characterized in that the sensor device 4 comprises several, in particular at least nine, emitters 5 and several, in particular at least four, detectors 6, preferably wherein several, in particular at least four, emitters 5 are assigned to each detector 6.
3. Examination apparatus according to aspect 1 or 2, characterized in that the emitters 5 and detectors 6 are arranged equidistantly and/or in a matrix with columns and rows, preferably the matrix having more than two columns and/or more than two rows, preferably the emitters 5 and detectors 6 being arranged alternately in the columns and rows in each case.
4. Examination apparatus according to one of the preceding aspects, characterized in that the limiting device 8 comprises a barrier 13 which is opaque to the radiation R emitted by the emitter 5, which is arranged between the emitter 5 and the detector 6 and limits an emission region 9 of the emitter 5 and/or a detection region 10 of the detector 6, so that the distance X of the border G of the sensing region 12 from the sensor device 4 is more than 0.5 mm and/or less than 5 mm.
5. Examination apparatus according to one of the preceding aspects, characterized in that the examination apparatus 1 has electrodes 15, 15A, 15B, 15C for recording a cardiogram KG, preferably wherein one of the electrodes 15, 15A, 15B, 15C is arranged in such a way that a paw 2 of the animal T can be positioned over the sensor device 4 in such a way that a cardiogram KG can be recorded by means of the electrode 15, 15A, 15B, 15C and at the same time the optical examination can be carried out by means of the sensor device 4.
6. Examination apparatus according to one of the preceding aspects, characterized in that the sensor device 4 has a cover 14 which is transparent for the radiation R emitted by the emitter 5, preferably wherein an electrode 15, 15A, 15B, 15C is arranged on the side of the cover 14 facing away from the emitter 5 and detector 6.
7. Examination apparatus according to aspect 6, characterized in that the electrode 15, 15A, 15B, 15C is arranged between the emitter 5 and the detector 6 and/or opposite the barrier 13 in a projection perpendicular to the cover 14 and/or to the plane defined by the emitters 5 and detectors 6 and/or in that the electrode 15, 15A, 15B, 15C is transparent to the radiation R emitted by the emitter 5.
8. Examination apparatus according to one of the preceding aspects, characterized in that an area density of the emitters 5 and/or detectors 6 and/or a common area density of the emitters 5 and detectors 6 is more than 0.5/cm², preferably more than 1/cm², in particular more than 2/cm², and/or less than 40/cm², preferably less than 20/cm², in particular less than 10/cm².
9. Examination apparatus according to one of the preceding aspects, characterized in that the limiting device 8 limits an emission angle 9A of the emitter 5 and/or a detection angle 10A of the detector 6 to less than 90°, preferably about 60°.
10. Examination apparatus according to one of the preceding aspects, characterized in that a height HB and width BB of the limiting device 8, a distance DB of the limiting device 8 from the emitter 5 and the detector 6 and a distance D of the emitter 5 from the detector 6 are matched to one another in such a way that an emission region 9 of the emitter 5 and/or a detection region 10 of the detector 6 overlap in such a way that the distance X of the boundary G of the detection area 12 from the sensor device 4 is more than 0.5 mm and/or less than 5 mm.
11. Examination apparatus according to one of the preceding aspects, characterized in that the sensor device 4 has more than 30, preferably more than 60, and/or less than 500, preferably less than 200, emitters 5.
12. Examination apparatus according to one of the preceding aspects, characterized in that the sensor device 4 comprises more than 20, preferably more than 40, and/or less than 500, preferably less than 200, detectors 6.
13. Examination apparatus according to one of the preceding aspects, characterized in that the emitters 5 are designed to emit radiation R of the same wavelength and/or that the detectors 6 are designed to detect at the same wavelength.
14. Examination apparatus according to one of the preceding aspects, characterized in that the emitter(s) 5 is/are designed to emit infrared radiation and/or radiation R with a wavelength of more than 900 nm and/or less than 1100 nm, preferably about 940 nm and/or 1050 nm.
15. Examination apparatus according to one of the preceding aspects, characterized in that the examination apparatus 1 is designed as a support, in particular a mat, for the animal T or the paw 2 or the body part, on which the animal T or the paw 2 or the body part is placed during the examination, the sensor device 4 being integrated in the support.
16. Examination apparatus 1, for the medical examination, in particular determination of a blood pressure BP, of an animal T having a paw 2, in particular an animal T from the subfamily of the Felinae, particularly preferably a domestic cat,
preferably wherein the examination apparatus 1 is designed according to one of the preceding aspects,
wherein the examination apparatus 1 is designed as a support for at least one paw 2 of the animal T,
wherein the examination apparatus 1 has a sensor device 4 for the optical examination of an arterial blood flow BF of the animal T, in particular for performing a photoplethysmography, wherein the sensor device 4 is designed for examination with electromagnetic radiation R in the infrared range, and/or
wherein the examination apparatus 1 has at least one detection element, preferably at least two electrodes 15, 15A, 15B, 15C, for recording a cardiogram KG, and/or
wherein the examination apparatus 1 has at least one tissue electrode, and/or wherein the examination apparatus 1 has or forms a scale 18.
17. Examination apparatus according to aspect 16, wherein the sensor device 4 has several emitters 5 and detectors 6, preferably wherein the several emitters 5 are designed for emission at the same wavelength and/or the detectors 6 are designed for detection at the same wavelength.
18. Examination apparatus according to aspect 17, wherein a detector 6 with one or more emitters 5 each forms a sensor 7, so that the sensor device 4 has several sensors 7, which form different measuring channels for the simultaneous recording of several curves comprising information about the arterial blood flow BF, in particular photoplethysmograms.
19. Examination apparatus according to one of the preceding aspects, wherein the electrodes 15, 15A, 15B, 15C are arranged at a distance of more than 5 cm and/or less than 20 cm
20. Examination apparatus according to one of the preceding aspects, wherein the examination apparatus 1 has a Wilson electrode 15C and two further electrodes 15A, 15B.
21. Examination apparatus according to one of the preceding aspects, wherein one of the electrodes 15, 15A, 15B, 15C is arranged in such a way that when a paw 2 of the animal T is positioned on the sensor device 4 to record a curve K comprising information about the arterial blood flow BF, in particular a photoplethysmogram, the electrode 15, 15A, 15B, 15C is simultaneously contacted.
22. Examination apparatus according to one of the preceding aspects, wherein the examination apparatus 1 is at least substantially flat, mat-like and/or plate-like.
23. Examination apparatus according to one of the preceding aspects, wherein the scale 18 and/or examination apparatus 1 is designed for body fat measurement, preferably wherein the examination apparatus 1 is designed to determine a blood pressure BP of the animal T taking into account the body fat measurement.
24. Examination apparatus according to one of the preceding aspects, wherein the examination apparatus 1 has a rest surface 3, wherein on the rest surface 3 an animal T from the subfamily of the Felinae, in particular a domestic cat, can be placed completely on the examination apparatus 1 and/or wherein the rest surface 3 has a width B of more than 20 cm, preferably more than 40 cm, and/or less than 80 cm, preferably less than 60 cm, and/or a length L of more than 40 cm, preferably more than 60 cm, and/or less than 120 cm, preferably less than 80 cm.
25. Examination apparatus according to one of the preceding aspects, wherein the examination apparatus 1 is designed or suitable for determining a diastolic blood pressure.
26. Use of an examination apparatus 1 according to one of the preceding aspects for medical examination, in particular determination of a preferably diastolic blood pressure BP, of an animal T having a paw 2, in particular an animal T from the subfamily of the Felinae, particularly preferably a domestic cat.
27. Method for the medical examination, in particular determination of a blood pressure BP, of an animal T having a paw 2, in particular an animal T from the subfamily of the Felinae, particularly preferably a domestic cat, wherein the animal T is positioned on an examination apparatus 1—which is in particular designed according to one of the preceding aspects—in such a way that a paw 2 of the animal T rests on a sensor device 4 of the examination apparatus 1, wherein a curve K comprising information about an arterial blood flow BF of the animal T, in particular a photoplethysmogram, is recorded by means of the sensor device 4, wherein, in order to record the curve K, a reflective measurement with electromagnetic radiation R in the infrared range is carried out, and/or
wherein a cardiogram KG of the animal T is recorded by means of the examination apparatus 1, and/or
wherein a signal is recorded by means of at least one tissue electrode, and/or where the animal T is weighed using the test apparatus 1.
28. Method according to aspect 27, wherein a curve feature, in particular a pulse transit time, is determined by means of the curve K and the blood pressure BP is preferably determined from the curve feature, in particular the pulse transit time, or on the basis thereof by means of a preferably empirically determined correlation function.
29. Method according to aspect 27 or 28, wherein the curve K and the cardiogram are recorded simultaneously, wherein the cardiogram KG is used to cut the curve K into curve sections KA corresponding to heartbeats.
30. Method according to one of the aspects 27 to 29, wherein a presence and/or positioning of the animal T is determined by means of the examination apparatus 1, in particular by evaluating signals measured with the sensor device 4, electrodes 15, 15A, 15B, 15C, a force sensor 18A and/or the balance 18.
31. Method according to one of the aspects 27 to 30, wherein a body fat measurement is carried out by means of the scale 18 and/or the examination apparatus 1, preferably wherein a blood pressure BP of the animal T is determined taking into account the body fat measurement.
32. Method according to one of the aspects 27 to 31, wherein a diastolic blood pressure BP is determined.
33. Method according to one of the aspects 27 to 32, wherein the examination apparatus 1 is designed according to one of the aspects 1 to 25.
34. Use of an examination apparatus 1 having a sensor device 4 for the optical examination of an arterial blood flow BF and at least one detection element, in particular electrodes 15, for recording a cardiogram KG, for determining a preferably diastolic blood pressure BP of an animal T which is freely movable relative to the sensor device 4 and/or the electrodes 15 or the detection element.
35. Use according to aspect 34, wherein the examination apparatus 1 is designed according to one of the aspects 1 to 25.
36. Use according to aspect 34 or 35, wherein the animal T has a paw 2, preferably wherein the animal T is an animal T from the subfamily of the Felinae, especially preferably a domestic cat.

What is claimed is:

1. An examination apparatus for medical examination of an animal, comprising:
a sensor device for the optical examination of an arterial blood flow of the animal,
wherein the sensor device has a plurality of emitters for emitting electromagnetic radiation and a plurality of detectors for detecting the radiation emitted by the emitters, and
wherein the emitters and detectors are arranged in a periodic structure.

2. The examination apparatus according to claim 1, wherein several emitters are associated with each detector.

3. The examination apparatus according to claim 2, wherein the emitters and detectors are arranged at least one of (a) equidistantly or (b) in a matrix with columns and rows, said matrix having at least one of (a) more than two columns or (b) more than two rows.

4. The examination apparatus according to claim 1, wherein the examination apparatus has at least one cardiogram detection element for recording a cardiogram, wherein one of the detection elements is arranged in such a way that an animal paw is positionable over the sensor device at a location that enables a cardiogram to be recorded by said at least one cardiogram detection element and wherein the optical examination can be carried out simultaneously by the sensor device.

5. The examination apparatus according to claim 1, wherein the sensor device has a cover that is transparent to the radiation emitted by the emitter, and wherein an electrode is arranged on a side of the cover facing away from the emitters and detectors.

6. The examination apparatus according to claim 1, further comprising an electrode which is at least one of (a) arranged in a projection perpendicular to a plane defined by the emitters and the detectors between the emitters and the detectors or (b) transparent to the radiation emitted by the emitter.

7. The examination apparatus according to claim 1, wherein the sensor device comprises at least one of (a) more than 30 emitters or (b) more than 20 detectors.

8. The examination apparatus according to claim 1, wherein the emitters emit radiation of the same wavelength and wherein the detectors detect at the same wavelength.

9. The examination apparatus according to claim 1, further comprising a support for the animal or the paw during the examination, and wherein the sensor device is integrated in the support.

10. An examination apparatus for medical examination of an animal, comprising:
a sensor device for optical examination of an arterial blood flow of the animal, wherein the sensor device has at least one emitter for emitting electromagnetic radiation and at least one detector for detecting the radiation emitted by the emitter,
wherein the sensor device has a limiting device which defines a border of a sensing region of the sensor device so that a distance of the border from the sensor device is at least one of (a) more than 0.5 mm or (b) less than 5 mm.

11. The examination apparatus according to claim 10, wherein the limiting device limits at least one of (a) an emission angle of the emitter or (b) a detection angle of the detector to less than 90°.

12. The examination apparatus according to claim 10, wherein the limiting device has a barrier that is opaque to the radiation emitted by the emitter, the barrier being arranged between the emitter and the detector and limiting at least one of (a) an emission region of the emitter or (b) a detection region of the detector, so that the distance of the border of the sensing region from the sensor device is at least one of (a) more than 0.5 mm or (b) less than 5 mm.

13. The examination apparatus according to claim 10, wherein a height and width of the limiting device, a distance of the limiting device from the emitter and the detector and a distance of the emitter from the detector are matched to one another in such a way that at least one of (a) an emission region of the emitter or (b) a detection region of the detector overlap such that the distance of a border of a sensing region from the sensor device is at least one of (a) more than 0.5 mm or (b) less than 5 mm.

14. The examination apparatus according to claim 1, wherein the examination apparatus is designed for determining diastolic blood pressure.

15. An examination apparatus for the medical examination of an animal having a paw, comprising:
a support for at least one paw of the animal, and
a sensor device for optical examination of arterial blood flow of the animal,
wherein at least one of:
(a) the sensor device is designed for examination with electromagnetic radiation in the infrared range, or
(b) the examination apparatus has at least two electrodes for recording a cardiogram.

16. The examination apparatus according to claim 15, wherein the sensor device comprises several emitters and detectors, wherein the several emitters are adapted to emit at the same wavelength and the detectors are adapted to detect the same wavelength.

17. The examination apparatus according to claim 15, wherein each detector has one or more emitters that forms a sensor of the sensor device, so that the sensor device has several sensors which are able to simultaneously record several curves comprising information about the arterial blood flow.

18. The examination apparatus according to claim 15, wherein one of the electrodes is arranged in such a way said one of the electrodes is simultaneously contacted when a paw of the animal is positioned on the sensor device for recording a curve comprising information about the arterial blood flow.

19. The examination apparatus according to claim 15, wherein the examination apparatus is at least one of (a) at least substantially flat, (b) mat-shaped or (c) plate-shaped.

20. The examination apparatus according to claim 15, further comprising a rest surface, wherein an animal from the subfamily of the Felinae can be completely placed on the rest surface.

21. A method of determining a blood pressure of an animal which is freely movable relative to one or more of a sensor device for optical examination of an arterial blood flow or a detection element for recording a cardiogram, the determining comprising:
placing the animal on an examination apparatus which comprises the sensor device for the optical examination of an arterial blood flow and the at least one detection element, and
determining blood pressure by examining the animal with the sensor device and the detection element.

22. The method according to claim 21, wherein the animal is an animal having a paw, wherein the paw is placed in a freely movable manner relative to one or more of the sensor device or a detection element, and wherein the determining of the blood pressure comprises medical examination of the paw with the sensor device and the detection element.
